SEQUENCE LISTING (34 sequences)

SEQ ID NO: 1 - DNA, 20 bp, Artificial sequence
  Description of the artificial sequence oligonucleotide probe
cctctgcagactactattac

SEQ ID NO: 2 - DNA, 20 bp, Artificial sequence
  Description of the artificial sequence Oligonucleotide target
gtaatagtagtctgcagagg

SEQ ID NO: 3 - DNA, 16 bp, Artificial sequence
  Description of the artificial sequence Oligonucleotide probe
atggcgtttagaaccc

SEQ ID NO: 4 - DNA, 16 bp, Artificial sequence
  Description of the artificial sequence Oligonucleotide probe
atgccgtatggaatcc

SEQ ID NO: 5 - DNA, 16 bp, Artificial sequence
  Description of the artificial sequence Oligonucleotide probe
atgtcgtgtcgaaacc

SEQ ID NO: 6 - DNA, 16 bp, Artificial sequence
  Description of the artificial sequence Oligonucleotide probe
atgacgtcttgaagcc

SEQ ID NO: 7 - DNA, 16 bp, Artificial sequence
  Description of the artificial sequence Oligonucleotide probe
acggcatttagtaccg

SEQ ID NO: 8 - DNA, 16 bp, Artificial sequence
  Description of the artificial sequence Oligonucleotide probe
acgccatatggtatcg

SEQ ID NO: 9 - DNA, 16 bp, Artificial sequence
  Description of the artificial sequence Oligonucleotide probe
acgtcatgtcgtaacg

SEQ ID NO: 10 - DNA, 16 bp, Artificial sequence
  Description of the artificial sequence Oligonucleotide probe
acgacatcttgtagcg

SEQ ID NO: 11 - DNA, 16 bp, Artificial sequence
  Description of the artificial sequence Oligonucleotide probe
agggcttttagcacca

SEQ ID NO: 12 - DNA, 16 bp, Artificial sequence
  Description of the artificial sequence Oligonucleotide probe
aggccttatggcatca

SEQ ID NO: 13 - DNA, 16 bp, Artificial sequence
  Description of the artificial sequence Oligonucleotide probe
aggtcttgtcgcaaca

SEQ ID NO: 14 - DNA, 16 bp, Artificial sequence
  Description of the artificial sequence Oligonucleotide probe
aggacttcttgcagca

SEQ ID NO: 15 - DNA, 16 bp, Artificial sequence
  Description of the artificial sequence Oligonucleotide probe
aaggcctttaggacct

SEQ ID NO: 16 - DNA, 16 bp, Artificial sequence
  Description of the artificial sequence Oligonucleotide probe
aagccctatgggatct

SEQ ID NO: 17 - DNA, 16 bp, Artificial sequence
  Description of the artificial sequence Oligonucleotide probe
aagacctcttggagct

SEQ ID NO: 18 - DNA, 16 bp, Artificial sequence
  Description of the artificial sequence Oligonucleotide probe
aagtcctgtcggaact

SEQ ID NO: 19 - DNA, 16 bp, Artificial sequence
  Description of the artificial sequence Oligonucleotide target
tcccgaaaatcgtggt

SEQ ID NO: 20 - DNA, 16 bp, Artificial sequence
  Description of the artificial sequence Oligonucleotide probe
gatcttcgccttactg

SEQ ID NO: 21 - DNA, 16 bp, Artificial sequence
  Description of the artificial sequence Oligonucleotide probe
gatcttcaccttactg

SEQ ID NO: 22 - DNA, 18 bp, Artificial sequence
  Description of the artificial sequence Oligonucleotide probe
gaaacaccaaagatgata

SEQ ID NO: 23 - DNA, 15 bp, Artificial sequence
  Description of the artificial sequence Oligonucleotide probe
gaaacaccgatgata

SEQ ID NO: 24 - DNA, 20 bp, Artificial sequence
  Description of the artificial sequence Oligonucleotide probe
cttctaattatttggtatgt

SEQ ID NO: 25 - DNA, 21 bp, Artificial sequence
  Description of the artificial sequence Oligonucleotide probe
cttctaattattttggtatgt

SEQ ID NO: 26 - DNA, 20 bp, Artificial sequence
  Description of the artificial sequence Oligonucleotide probe
gagttcttctaattatttgg

SEQ ID NO: 27 - DNA, 21 bp, Artificial sequence
  Description of the artificial sequence Oligonucleotide probe
gagttcttctaattattttgg

SEQ ID NO: 28 - DNA, 21 bp, Artificial sequence
  Description of the artificial sequence Oligonucleotide probe
ttttagagttcttctaattat

SEQ ID NO: 29 - DNA, 22 bp, Artificial sequence
  Description of the artificial sequence Oligonucleotide probe
ttttagagttcttctaattatt

SEQ ID NO: 30 - DNA, 20 bp, Artificial sequence
  Description of the artificial sequence Oligonucleotide target
ctcagtaaggcgaagatctt

SEQ ID NO: 31 - DNA, 22 bp, Artificial sequence
  Description of the artificial sequence Oligonucleotide target
aatatcatctttggtgtttcct

SEQ ID NO: 32 - DNA, 34 bp, Artificial sequence
  Description of the artificial sequence Oligonucleotide target
gaacataccaaataattagaagaactctaaaaca

SEQ ID NO: 33 - DNA, 19 bp, Artificial sequence
  Description of the artificial sequence Oligonucleotide probe
ttttttttttttttttttt

SEQ ID NO: 34 - DNA, 36 bp, Artificial sequence
  Description of the artificial sequence Oligonucleotide target
tcccgaaaatcgtggtaaaaaaaaaaaaaaaaaaaa

(19) United States
(12) Patent Application Publication
(10) Pub. No.: US 2003/0215891 A1
(43) Pub. Date: Nov. 20, 2003

(54) Method for the qualitative and/or quantitative detection of molecular interactions on probe arrays

(76) Inventors: Ralf Bickel, Jena (DE); Ralf Ehricht, Jena (DE); Thomas Ellinger, Jena (DE); Eugen Ermantraut, Jena (DE); Thomas Kaiser, Jena (DE); Torsten Schulz, Jena (DE); Gerd Wagner, Jena (DE)

Correspondence Address: KENYON & KENYON, ONE BROADWAY, NEW YORK, NY, 10004, US

(21) Appl. No.: 10/331,109

(22) Filed: Dec. 27, 2002

Related U.S. Application Data
(63) Continuation of application No. PCT/EP01/07575, filed on Jul. 2, 2001.

(30) Foreign Application Priority Data
Jul. 1, 2000 (DE) 100 33 334.6

(51) Int. Cl. G01N 33/53; G01N 33/537; G01N 33/543; C12Q 1/68

(52) U.S. Cl. 435/7.5; 435/7.92; 435/6

(57) ABSTRACT

The invention relates to a method for qualitatively and/or quantitatively detecting certain molecular targets using probe arrays. The inventive detection method comprises a reaction which delivers a product with a particular solubility product, this solubility product causing the precipitation or the formation of a precipitate of the product on an array element of the probe array on which an interaction has taken place between the probe and the target.

The invention relates to a device and a method for the qualitative and/or quantitative detection of defined molecular targets with the help of probe arrays.

Biomedical tests are often based on the detection of the interaction between a molecule which is present at a definite position and in a known quantity (the molecular probe) and the molecule or molecules which are to be detected (the molecular target). Modern tests are usually performed in parallel with several probes in one sample (D. J. Lockhart, E. A. Winzeler; Genomics, gene expression and DNA arrays; Nature 2000, 405, 827-836). Conventionally, the probes are then immobilised in a prescribed manner on a suitable matrix, such as that described in WO 00/12575 (see e.g. U.S. Pat. No. 5,412,087, WO 98/36827) or are produced synthetically (see e.g. U.S. Pat. Nos. 5,143,854, 5,658,734, WO 90/03382).

An interaction of this sort is normally detected as follows: The probe or probes are attached to a defined matrix in a prescribed manner. A solution of the targets is brought into contact with the probes and incubated under defined conditions. As a result of the incubation, a specific interaction between probe and target develops. The resulting bond is clearly more stable than the binding of molecules for which the probe is unspecific. The system is then washed with appropriate solutions, so that those molecules are removed which are not specifically bound.

Many procedures are used today to detect the interaction between target and probe; some of these will now be described:

E. Lidell and I. Weeks, Antibody Technology, BIOS Scientific Publishers Limited, 1995, describe the labelling of the target with a dye or with a fluorescent dye and the detection of this with a photometer or fluorometer, respectively.

F. Lottspeich, H. Zorbas, Bioanalytik, Spektrum Akademischer Verlag, Heidelberg, Berlin, 1998, also describe the optical detection of the fluorescence of targets which have been labelled with a fluorescence marker.

In Nature Biotechnology 1998, 16, 725-727, the detection of complexes between target and probe by mass spectroscopy is described. Mass sensitive procedures such as surface plasma resonance are also used (J. M. Brockman et al., A multistep chemical modification procedure to create DNA arrays on gold surfaces for the study of protein-DNA interactions with surface plasma resonance imaging, J. Am. Chem. Soc. 1999, 121, 8044-8051). U.S. Pat. No. 5,605,662 discloses a procedure for the direct electrical detection of the interaction. In DE 19543232 the labelling of the target with detection beads is described; the presence of these can be detected optically after the interaction between the target and probe.

EP 0 063 810 discloses a procedure in which targets in the form of antigens or immunoglobulins are immobilised on a solid porous substrate. Their identity and quantity is then examined with conventional immunological techniques, particularly ELISA.

Various different technical approaches have been described for the detection of molecular interactions with the help of arrays of probes. Classical systems are based on a comparison of the intensity of fluorescence of target molecules which have been labelled with fluorophores and then selectively excited at specific wavelengths. Various technical solutions are possible for this, which have different optical construction and different components. The problems and limitations of these approaches result from the signal noise (the background), which is largely the result of effects such as bleaching and quenching of the dyes used, autofluorescence of the media, elements in the assembly and optical components and scatter, reflection and external light in the optical system.

As a result of this, the technical demands are high for the assembly of highly sensitive fluorescence detectors for the qualitative and quantitative comparison of probe arrays. Specially adapted systems are particularly required for screening of intermediate or high throughput, as this requires a certain degree of automatisation.

CCD based detectors are known for the optimisation of standard π-fluorescence assemblies, which can discriminate optical effects such as scatter and reflection from the excitation of the fluorophore in the dark field by incident or transmitted light (see e.g. C. E. Hooper et al., “Quantitative Photon Imaging in the Life Sciences Using Intensified CCD Cameras”, Journal of Bioluminescence and Chemiluminescence (1990), p. 337-344.). The assay is then mapped with high resolution optics, either under illumination or screening. The use of multispectral sources of illumination allows a relatively simple approach to different fluorophores, by using different combinations of excitation filters. It is however a disadvantage that autofluorescence and systemic optical effects, such as the homogeneity of the illumination, require complicated illumination optics and filter systems.

As an example, the confocal scanning system described in U.S. Pat. No. 5,304,810 is based on selection of the fluorescence signals along the optical axis with the help of two pinholes. This either makes adjusting the sample difficult or necessitates a powerful autofocussing system. The technical solution of such systems is highly complex. The components required, such as lasers, pinholes, perhaps cooled detectors, such as for example PMT, avalanche diodes or CCD, together with complex and highly exact mechanical translation elements and optics, must be mutually optimised and integrated, which requires a great deal of effort (see for example U.S. Pat. Nos. 5,459,325; 5,192,980; 5,834,758). The degree of minituarisation and the price are limited by the multitude and functionality of the components.

At the present time, analyses based on probe arrays are usually measured on the basis of optical fluorescence (see A. Marshall and J. Hodgson, DNA Chips: An array of possibilities, Nature Biotechnology, 16, 1998, 27-31; G. Ramsay, DNA Chips: State of the Art, Nature Biotechnology, 16, January 1998, 40-44). The high signal background is a disadvantage of this detection procedure, which restricts the accuracy. Further disadvantages are the technical demands, which may be high, and the expenses of the detection procedure.

There is therefore a requirement for highly integrated arrays, with which the interaction between probes and targets can be very accurately measured, qualitatively and/or quantitatively, and with low technical expenditure.

An increase in selectivity and the access to alternative components provide the motive for the establishment of alternative imaging technologies, such as fluorescence polarisation and time-resolved fluorescence for solid-bound assays. However, these solutions are only available as concepts, particularly for highly integrated assays. The effect of the rotation of the axis of polarisation by a fluorophore which has been excited with polarised light is used for quantification in the microtitre format. There have also been attempts to assemble cheap systems with a high throughput (HTS systems) by using an appropriately modified polymer film as polarisation filter (see I. Gryczcynski et al., Polarisation sensing with visual detection, Anal. Chem. 1999, 71, 1241-1251). Adaptation to microassays is however difficult with the available light intensities and detectors. A system of this type would require the integration of light sources (e.g. laser, LED, high pressure lamps), polarisation filters (perhaps coated polymer films) and detectors (CCD-, CMOS-camera); no solution is known at present.

Newer developments use the fluorescence of inorganic materials, such as lanthamides (M. Kwiatowski et al., Solid-phase synthesis of chelate-labelled oligonueleotides: application in triple-colour ligase-mediated gene analysis, Nucleic Acids Research, 1994, 22, 13) and quantum dots (M. P. Bruchez et. al., Semiconductor Nanocrystals as Fluorescent Biological Labels, Science 1998, 281, 2013). The exploitation of the specific fluorescence lifetime of fluorescence in the ns range for its selective quantification is very demanding and is not used commercially, in spite of the specificity of the site-resolved application. Dyes like lanthamide gelates, with long emission lifetimes in the μsec range, require conversion of the dyes into the mobile phase, so that site-specific detection is not possible.

Microparticles are familiar from their use in television tubes (see F. van de Rijke et al., Up-Converting Phosphors: A New Reporter Technology for Nucleic Acid Microarrays, European EC Meeting on Cytogenetics (2000) Bari, Italy) and their use as biological markers has great potential in detection technology, with respect to sensitivity and minituarisation, particularly as excitation light sources are used in data transfer (980 nm diode laser). However, this technology is not commercially available for the detection of target/probe interactions in arrays. A detector would include components for light emission (e.g. laser, LED, high pressure lamps), a system for modulating the excitation and detection light (e.g. chopper blades, electronic shutter) and detection of the time-delayed signal (e.g. CCD-, CMOS-camera). The fundamental difficulty however appears to be the low compatibility between the particles and biological samples.

In contrast to the use of probe arrays, the use of arrays with immobilised targets has the disadvantage in principle that, for each analysis, an array with the material to be investigated must be produced, so that known probes can be combined in one batch. This greatly restricts the diagnostic use, as the arrays have to be prepared frequently. As the material is usually of biological origin, differences between batches are inevitable. The use of porous substrates restricts the maximum attainable resolution of the arrays produced, as the applied fluid can spread laterally. With the present technique of deposition, the individual elements on the porous materials can hardly be reduced to lower than 200 μm.

It is therefore the object of the present invention to overcome these problems in the state of the art, particularly those resulting from the complex structure of the detection system, the high signal background, particularly from the bleaching of the signal and the inadequate compatibility of the assay with the test system.

In particular, it is an object of the present invention to provide a method or device with which molecular interactions between probes and targets on the probe array can be detected with high accuracy, simply and cheaply, both qualitatively and/or quantitatively.

It is a further object of the present invention to achieve high dynamic resolution with the detection, so that weak probe/target interactions may be reliably detected in the presence of strong signals.

These and other objects of the present invention are solved by the embodiments characterised in the claims.

Surprisingly, it has now been found that molecular interactions between probe molecules (referred to as probes below) and target molecules (referred to as targets below) can be detected with high accuracy on probe arrays with a simple and cheap technique. The detection is carried out by the method according to the present invention, using a reaction which gives a product with a given solubility product, which results in a precipitate of the product on an array element of the probe array on which the interaction between probe and target has occurred.

The bound targets are preferably supplied with a label which catalyses the reaction of a soluble substrate to form a precipitate of low solubility on the array element on which the probe/target interaction has occurred, or which acts as crystallisation seed for the conversion of a soluble substrate to a precipitate of low solubility on the array element on which the probe/target interaction has occurred.

The use of probe arrays on non-porous carriers allows the simultaneous qualitative and quantitative analysis in this way of many probe/target interactions. Individual probe sizes of ≦1000 μm, preferred ≦100 μm, especially preferred ≦50 μm can then be attained.

The use of enzyme labelling is known in immunocytochemistry and in immunological microtitre plate-based tests (see E. Lidell and I. Weeks, Antibody Technology, BIOS Scientific Publishers Limited, 1995). For example, an enzyme can catalyse the conversion of a substrate into a product of low solubility, which is usually coloured. Another possible way of detecting molecular interactions in arrays is by using metal labelling. Colloidal gold or a defined gold cluster is then coupled with the target, optionally through an intermediate molecule such as streptavidin. The product is then enhanced by subsequent reaction with a more reactive metal such as silver.

The relative quantification of the concentration of the bound target on a probe array by detecting the precipitate is carried out, in accordance with the invention, by a method comprising the detection of the concentration of the label which is coupled to the target, wherein the label either catalyses the reaction of a soluble substrate to form a precipitate of low solubility on the array element on which the probe/target interaction has occurred, or which serves as crystallisation seed for reactions of this sort. For example, in the case of HPLC-purified oligonucleotide probes labelled with nanogold, the ratio of bound target to gold particles is 1:1. In other embodiments of the present invention, it can amount to a multiple or to a fraction of this.

The concentration of the marker or label coupled to the target (c(L)) is related to the concentration of the precipitate (c(P)) on the array element according to the following equation:

c(L)=[F*c(P)]/t,

where F is a curve function which characterises the time course of the precipitation reaction and t is the time.

F can be determined from the time course of the reaction. In the case that the time course can be described as a linear function (F=constant), an unambiguous correlation is possible between the formed precipitate and the concentration of bound target molecules, as c(P)/t is then a measure of c(L) and therefore also of the concentration of the labelled target. An unambiguous relative determination of the target concentration which is bound to the corresponding array elements is consequently only possible if the time course of the precipitation reaction is known.

The conventional procedure is that, a certain time after the interaction of the targets with the probes arranged on the array and after the beginning of the reaction which leads to a precipitation on the array elements on which the interaction has occurred, a picture or image is taken and concentrations are assigned to the measured grey values, which depend on the degree of precipitation. However, this procedure only leads to satisfactory values for each array element in a very narrow concentration range and is therefore problematical for the evaluation of the specificity of interactions. The reason for this is that the formation of the precipitate is highly non-linear. In particular, the time course of the precipitation includes an exponential rise with time, followed by a saturation plateau. Only grey values from the phase of exponential increase allow a correlation with the quantity of bound target. The saturation plateau for the array element is dependent on the relevant probe/target interaction and is therefore reached at a different time for each element of the array. This militates against quantification after the end of the precipitation reaction. It is impossible to design the experimental parameters in such a way that the saturation plateau is reliably attained in no member of the array, as the rate of the reaction strongly depends on temperature, light, salt concentration, pH and other factors.

If only one picture is taken, there can therefore be no guarantee that the precipitation is in the exponential phase of dependency of the precipitate formation with time in all array elements. This leads to a distorted comparison between signal intensities, such as grey values, from array elements in which the precipitation reaction is already in the saturation plateau and signals from arrays which are still in the exponential phase of the precipitation reaction.

To overcome the above disadvantages, the present invention provides a method for the qualitative and/or quantitative detection of targets in a sample by molecular interactions between probes and targets on probe arrays, including the following steps:

a) Preparation of a probe array with probes immobilised at defined sites;

b) Interaction of the target with the probes arranged on the array of probes;

c) Performance of a reaction which leads to a precipitate on the array elements on which the interaction occurs;

d) Detection of the time course of the formation of the precipitate on the array elements in the form of signal intensities;

e) Determination of a virtual signal intensity for an array element on the basis of a curve function which describes the formation of the precipitate as a function of time.

The following definitions are used to describe the present invention:

In the context of the present invention, a molecular probe means a molecule which is used to detect other molecules as a result of a certain and characteristic binding behaviour or defined reactivity.

In the context of the present invention, a probe array means an array of molecular probes on a surface, where the position of each probe is determined separately.

In the context of the present invention, an array element means a defined area on a surface which is intended for the deposition of a molecular probe. The sum of all occupied array elements is the probe array.

In the context of the present invention, a microtitre plate means an array of reaction vessels in a defined grid, which allows the automatised performance of a variety of biological, chemical and clinical chemical tests.

In the context of the present invention, a target means the molecule which is to be detected with the molecular probe.

In the context of the present invention, HTS (Engl.: high throughput screening) means a systematic search with a high throughput for active substance.

In the context of the present invention, a substrate means a molecule or combination of molecules which are dissolved in the reaction medium and which are locally deposited as a result of the action of a catalyst or a crystallisation seed and a reductant.

In the context of the present invention, a carrier means a solid on which the probe array is assembled.

In the context of the present invention, a label means as group which is coupled with the target and which catalyses the reaction of a soluble substrate to a precipitate of low solubility or which acts as a seed of crystallisation to convert a soluble substrate to a precipitate of low solubility.

In the context of the present invention, a virtual signal intensity means a value which quantifies the interaction between probe and target on an array element and thereby the quantity of bound target, and which is determined on the basis of a curve function which describes the formation of precipitate as a function of time.

An essential characteristic of a method according to the invention is the determination of a virtual signal intensity for an array element in dependence on the time course of the formation of the precipitate. In accordance with the invention, the formation of the precipitate on the array element as a function of time is preferably described as a curve function, on the basis of which a virtual signal intensity is determined. Because of the consideration of the time course of the formation of the precipitate, this virtual signal intensity is an undistorted measure of the quantity of bound target.

In particular, in accordance with the invention partial sections or the whole length of the time course of the formation of the precipitate may be described as a regression line. In this embodiment, the virtual signal intensity is described in dependency on the gradient of the regression line. The gradient is a direct measure of the concentration of the bound target, i.e. the greater the gradient of the regression line, the more target is bound. If all array elements are monitored under the same conditions, the increase in precipitate formation over time on each array element is characteristic of the concentration and for the current experiment, normalised to the dominant conditions. This then guarantees exact determination of the relative quantities of bound targets.

In a particularly preferred embodiment of the present invention, the regression line in the phase of the exponential increase in precipitate formation with time is determined for one array element.

In an embodiment of the present invention, the regression line corresponds to a tangent to the curve function with which the formation of the precipitate as a function of time can be described, drawn through the point of inflection. The point of inflection of the curve function is determined from the maximum of the first derivative of the curve function.

In an alternative embodiment of the present invention, the regression line is determined by connecting with a line the vertexes of the curve function with which the formation of the precipitate with time can be described. The vertexes of the curve functions are determined from the maxima of the second derivative of the curve function.

The determination of the virtual signal intensity for each array element depending on the time course of precipitate formation, followed by conversion of these virtual signal intensities into an analogue image, leads to expansion of the dynamic range of measurement, i.e. the range in which detection is possible is multipled. An extension of the dynamics of the measurement is possible, as the depth of colour of the detector system is no longer decisive, but the time course of the deposition of the precipitate on the surface of each element of the probe array. By evaluating the increase in the precipitation reaction, it is possible to determine a virtual signal intensity of grey value distribution and thus to extend the dynamic range.

The procedure in accordance with the invention has the further advantage that detection systems can be used which are simple and also cheap. For example, a camera with only 8 bits, i.e. 256 grey values, can be used to determine the depth of grey. After calculation and virtual mapping, this gives a real depth of focus of 24 bits (16777216 grey values) or of 48 bits (33554432 grey values). This then allows clearly improved possibilities for the simultaneous detection of weak and strong interactions between targets and the probes on the probe array.

In a preferred embodiment of the present invention, a reference target of known concentration is present in the sample to be examined, which interacts with at least one probe of the probe array. The virtual signal intensity which corresponds to this probe/reference target interaction is determined in dependency on the increase in formation of precipitate with time and serves as reference for the quantification of the other target concentrations, in accordance with their virtual signal intensities, which are evoked by the probe/target interactions, relative to the reference target concentration.

The targets to be examined can be in any type of sample, preferably in a biological sample. The targets are preferably isolated, purified, copied and/or amplified before their detection and quantification by the method according to the present invention.

The probe array used in the context of the present invention, with immobilised probes in defined sites, is produced by conventional methods. In accordance with the present invention, a probe array includes a carrier which permits the formation of probe arrays on its surface. A carrier of this sort can be made of materials selected from the group consisting of glass, filters, electronic devices, polymers, metallic materials and similar, or combinations of these. The array preferably includes defined sites, so-called array elements, which are particularly preferred to be in a certain pattern, where each array element only contains one type of probe.

In a further embodiment of the present invention, the intensity of the signals used to detect the time course of the formation of precipitate in the array elements are recorded every minute, preferably every 30 seconds, more preferably every 10 seconds. Other time intervals for recording the signals are also conceivable, with the condition that the time dependency of the formation of the precipitate can be determined unambiguously and that, for example, the gradient of the regression line in the exponential phase can be derived as a measure for the concentrations of the bound targets.

The virtual signal intensity for an array element is determined, for example, by multiplication of the detected signal intensity at a certain time point, preferably the signal intensity of the last measurement, by the gradient of the regression line determined for the array element and by the duration of measurement up to this time point. In this embodiment it is evidently necessary for relative quantification that the time point for detection of the signal intensity is identical for all array elements.

A further condition for the relative quantification of the concentration of the bound target on the probe array by detection of a precipitate in accordance with the method in the invention is that the target is supplied with labels which catalyse the reaction of a soluble substrate to a poorly soluble precipitate on the array element on which the probe/target interaction has occurred or which serve as crystallisation seed for reactions of this sort.

In one embodiment of the present invention, the targets can be directly supplied with labels of this sort.

Alternatively, direct labelling of the target is dispensed with and the labelling is carried out by sandwich hybridisation or sandwich reactions with the probe which interacts with the target and a labelled compound. Examples of a procedure of this sort are:

Sandwich hybridisation with a labelled oligonucleotide with a sequence which is complementary to the target sequence,

Sandwich hybridisation of labelled oligonucleotides which hybridise in the chain form with the target sequence: in the context of the present invention, hybridising with the chain form of the target sequence means that there is a group of labelled oligonucleotides of which at least one exhibits complementarity both to the target sequence and to another oligonucleotide. The other oligonucleotides are then self-complementary or mutually complementary to each other, so that a chain of labelled oligonucleotides arises during hybridisation which is bound to the target sequence.

Sandwich hybridisation with an oligonucleotide which is complementary to the target sequence and which is coupled to a multiply labelled structure, such as a dendrimer, as described for example in WO 99/10362.

A further preferred possibility for the coupling of the target with a label is the synthetic or enzymatic introduction of a homopolymeric region, for example a polyA sequence, to the target, resulting in the formation of a continuous sequence, as for example described in U.S. Pat. No. 6,103,474. In this embodiment, the labelling is carried out preferably by sandwich hybridisation with a labelled oligonucleotide which is complementary to the homopolymer sequence, with the variations described above.

In another preferred embodiment of the present invention, signal amplification is carried out by amplification of sections of the homopolymer sequence which has been added to the target, with the simultaneous incorporation of labelled bases. An especially preferred embodiment is to use an RCA mechanism with a circular single-stranded template, which exhibits complementarity to the homopolymer sequence.

The following Table 1, which does not claim to be a complete list, gives a summary of the series of possible reactions which are suitable to cause a precipitate on array elements on which an interaction between target and probe has occurred:

TABLE 1

| Catalyst or Crystallisation | |
|---|---|
| Seed | Substrate |
| Horseradish Peroxidase | DAB (3,3′-Diaminobenzidine) |
| | 4-CN (4-Chlor-1-Napthol) |
| | AEC (3-Amino-9-Ethylcarbazole) |
| | HYR (p-Phenylendiamine-HCl and Pyrocatechol) |
| | TMB (3,3 ′,5,5 ′-Tetramethylbenzidine) |
| | Naphthol/Pyronine |
| Alkaline Phosphatase | Bromchlorindoylphosphate (BCIP) and Nitrotetrazolium blue |
| | (NBT) |
| Glucose Oxidase | t-NBT and m-PMS |
| | (Nitrotetrazolium blue chloride and Phenazine methosulphate |
| Gold Particles | Silver nitrate |
| | Silver tartrate |

The labelling of biological samples with enzymes or gold, particularly nanocrystalline gold, has been adequately described (see i.a. F. Lottspeich and H. Zorbas, Bioanalytik, Spektrum Akademischer Verlag (Springer Academic Press), Heidelberg, Berlin, 1998; E. Lidell and I. Weeks, Antibody Technology, BIOS Scientific Publishers Limited, 1995).

Other possibilities for the detection of probe/target interactions with insoluble precipitates, with the procedure in accordance with the invention, are described in: Immunogold-Silver Staining, Principles, Methods and Applications, Eds.: M. A. Hayat, 1995, CRC Press; Eur J Immunogenet February-April 1991;18(1-2):33-55 HLA-DR, DQ and DP typing using PCR amplification and immobilized probes. Erlich H, Bugawan T, Begovich AB, Scharf S, Griffith R, Saiki R, Higuchi R, Walsh PS. Department of Human Genetics, Cetus Corp., Emeryville, Calif. 94608; Mol Cell Probes June 1993;7(3):199-207 A combined modified reverse dot-blot and nested PCR assay for the specific non-radioactive detection of Listeria monocytogenes. Bsat N, Batt C A.

Department of Food Science, Cornell University, Ithaca, N.Y. 14853. Immunogenetics 1990;32(4):231-41 Erratum in: Immunogenetics 1991;34(6):413 Rapid HLA-DPB typing using enzymatically amplified DNA and nonradioactive sequence-specific oligonucleotide probes. Bugawan T L, Begovich A B, Erlich H A. Department of Human Genetics, Cetus Corporation, Emeryville, Calif. 94608. Hum Immunol December 1992;35(4):215-22 Generic HLA-DRB1 gene oligotyping by a nonradioactive reverse dot-blot methodology. Eliaou J F, Palmade F, Avinens O, Edouard E, Ballaguer P, Nicolas J C, Clot J. Laboratory of Immunology, Saint Eloi Hospital, CHU Montpellier, France. J Immunol Methods Nov. 30, 1984;74(2):353-60 Sensitive visualization of antigen-antibody reactions in dot and blot immune overlay assays with immunogold and immunogold/silver staining. Moeremans M, Daneels G, Van Dijck A, Langanger G, De Mey J. Histochemistry 1987;86(6):609-15 Non-radioactive in situ hybridization. A comparison of several immunocytochemical detection systems using reflection-contrast and electron microscopy. Cremers A F, Jansen in de Wal N, Wiegant J, Dirks R W, Weisbeek P, van der Ploeg M, Landegent J E.

In the context of the present invention, possible variants for the detection of probe/target interactions with insoluble precipitates include the following:

In one embodiment of the present invention, the targets are supplied with a catalyst, preferably an enzyme, which catalyses the conversion of a soluble substrate into an insoluble product. The reaction which leads to the formation of a precipitate on the array elements is, in this case, the conversion of a soluble substrate into an insoluble product in the presence of a catalyst which is coupled to the target, preferably an enzyme. The enzyme is preferably selected from the group containing horseradish peroxidase, alkaline phosphatase and glucose oxidase. The soluble substrate is preferably selected from the group containing 3,3′-diaminobenzidine, 4-chlor-1-naphthol, 3-amino-9-etllylcarbazole, p-phenylendiamine-HCl/pyrocatechol, 3,3′, 5,5′-tetramethylbenzidine, naphthol/pyronine, bromchlorindoylphosphate, nitrotetraazolium blue and phenazine methosulphate. For example, a colourless soluble hydrogen donor, such as 3,3′-diaminobenzidine, is converted into an insoluble coloured product in the presence of hydrogen peroxide. The enzyme horseradish peroxidase transfers hydrogen ions from the donors to hydrogen peroxide, forming water.

In a preferred embodiment of the present invention, the reaction which leads to the formation of a precipitate on the array elements is the formation of a metallic precipitate. It is particularly preferred if the reaction which leads to the formation of a precipitate on the array elements is the chemical reduction of a silver compound, preferably silver nitrate, silver lactate, silver acetate or silver tartrate, to silver. The preferred reductants are formaldehyde and/or hydroquinone.

It is particularly preferred if the precipitation of the metallic compound occurs in the presence of targets labelled with metal clusters or colloidal metal particles, particularly gold clusters or colloidal gold particles. In other words, in this case the metal clusters or colloidal metal particles are the labels coupled to the targets. For example, silver nitrate is converted into elemental silver, during which process silver ions from the solution are deposited on gold as crystallisation seed and are then, in a second step, reduced with the help of a reductant, such as formaldehyde. An insoluble precipitate of elemental silver results in this way.

In an alternative embodiment, the precipitation of the metallic compound occurs in the presence of polyanions which are coupled with the target. If the target itself is not a polyanion, there is the possibility of using the polyanion as crystallisation seed. For example, the target labelled with a polyanion is exposed to a solution of silver nitrate. The silver cations are then selectively accumulated on the polyanion. Silver ions are then converted into elemental silver with a reductant.

The coupling of the enzymes or the catalysts or the colloidal metal particles or the polyanions to the targets can either happen directly or through anchor molecules which are coupled to the target. It is not necessary in principle to equip the target directly with the labels described above. It is possible to couple the labels in a second step, using anchor molecules such as streptavidin which are themselves coupled to the target.

A conjugate consisting of the relevant catalyst or crystallisation seed and a specific binding partner for the anchor molecule also allows the performance of the procedures described above. The reaction which leads to the formation of a precipitate on the array elements is then the binding of a specific binding partner to an anchor molecule which is coupled to the target.

Binding partner/anchor molecule pairs are preferably selected from the group of biotin/avidin or streptavidin or antibiotin antibodies, digoxigenin/antidigoxigenin immunoglobul in, FITC/anti-FITC immunoglobulin and DNP/anti-DNP immunoglobulin.

In each of the embodiments described above, a soluble catalyst is converted catalytically into an insoluble precipitant product. Because of the nearness of the surface, the product is deposited directly on the surface and forms a solid precipitate which is not removed by washing in various ways.

It is also possible, in the context of the present invention, to couple the labels, particularly the enzymes, metal clusters, colloidal metal particles or polyanions, to the targets, either before, during or after the interaction with the probes.

In a further preferred embodiment of the present invention, the interaction between the target and the probe is hybridisation between two nucleotide sequences. The hybridisation of the targets with the probes in the probe array is carried out according to one of the known standard protocols (see i.a. Lottspeich and Zorbas, 1998). The resulting hybrids can be stabilised by covalent binding, for example with psoralene intercalation and subsequent “crosslinking”, or, as described in U.S. Pat. No. 4,599,303, by non-covalent binding, for example by binding of intercalators.

After the hybridisation of the target with probes in the probe array or the labelling of the hybridised target, a washing step is usually carried out, with which the non-specific and therefore more weakly bound components are removed.

As an alternative, the interaction between the target and the probe is a reaction between an antigenic structure and the corresponding antibody, or a hypervariable region of this, or a reaction between a receptor and the corresponding ligand.

The binding or recognition of the target by specific probes is usually a spontaneous non-covalent reaction under optimal conditions. This also includes non-covalent chemical bonds. The composition of the medium and other chemical and physical factors influences the rate and strength of the binding. For example, in the recognition of nucleic acids, low stringency and higher temperatures lower the rate and strength of the binding between two strands which are not perfectly complementary. Optimisation of the binding conditions is also required for antigen/antibody or ligand/receptor interactions, although the binding conditions are usually less specific.

In one embodiment of the present invention, the presence of a precipitate on an array element is carried out by reflection, absorption or diffusion of a light beam, preferably a laser beam or a light-emitting diode, by the precipitate. Because of its granular form, the precipitate modifies the reflection of the light beam. The precipitate also leads to marked light diffusion, which can be recorded with conventional detection systems. If the precipitate, such as a silver precipitate, appears as a dark surface, the absorption of light can be detected and recorded. The resolution of the detection depends on the number of pixels in the camera.

For example, the detection of the regions which are intensified by the specific reaction can be carried out with a very simple optical structure in transmitted light (contrast with shadowing) or incident light (contrast with reflection). The detected intensity of the shadowed regions is directly proportional to the degree of occupation with labels such as gold particles and the state of nucleus formation of the particles.

If a precipitate is used which is electrically conducting or which has a dielectric constant different from the environment, the reaction may also be detected electrically in an alternative embodiment.

The electrical measurements can be on the basis of conductivity measurements with microelectrode arrays or with an array of microcapacity sensors or with potential measurements by arrays of field effect transistors (FET arrays). If the conductivity is measured with microelectrodes, the change in the electrical resistance between two electrodes is followed with a deposition reaction (E. Braun, Y. Eichen, U. Sivan, G. Ben-Yoseph, Nature, 775, vol 391, 1998). If dielectric measurements are made with microcapacity sensors, the change in the capacity of two apposed electrodes is measured (M. Madou, Fundamentals of Microfabrication, CRC Press, Boca Raton, 1997). If potentials are measured with FET arrays, the change in the potential on the surface of the sensor is measured (M. Madou, Fundamentals of Microfabrication, CRC Press, Boca Raton, 1997).

If a substrate is used which is radioactive or radioactively labelled, the presence of a precipitate on an array element can be detected with autoradiography, fluorography and/or indirect autoradiography. In autoradiography, a surface which is covered with an irradiating precipitate is brought into contact with an X-ray film. In fluorography, a surface which is in contact with an irradiating precipitate is overlaid with fluorescent chemicals such as sodium salicylate, which convert the radioactive irradiation energy into fluorescence. In indirect autoradiography with intensifier screens, a surface which is covered with a precipitate which emits β- radiation is laid on an intensifier screen, which converts the irradiation into blue light (see F. Lottspeich, H. Zorbas, see above). However, detection procedures based on radioactivity are often not desired, because of the risks to health and the safety regulations which therefore have to be fulfilled.

In a further alternative embodiment of the present invention, the precipitate on the array element is detected with scanning electron microscopy, electron probe microanalysis (EPMA), magneto-optic Kerr microscopy, magnetic force microscopy (MFM), atomic force microscopy (AFM), measurement of the mirage effect, scanning tunnelling microscopy (STM) and/or ultrasound reflection tomography.

Detection of the reaction with SEM and/or EPMA is almost independent of the type of the substrate. In scanning electron microscopy (SEM), a focussed electron beam scans the sample (J. Goldstein et al. Scanning Electron Microscopy and X-Ray Microanalysis, Plenum, New York, 1981). In electron probe microanalysis (EPMA), the secondary processes which are triggered by a focussed electron beam are used for site-resolved analysis (J. Goldstein et al. Scanning Electron Microscopy and X-Ray Microanalysis, Plenum, New York, 1981).

If a substrate is used which is magnetic or which is labelled with magnetic particles, the reaction can be detected with magneto-optic Kerr microscopy or MFM. In magneto-optic Kerr microscopy, the rotation by magnetic field of the plane of polarisation of the light (Kerr-Faraday effect) is exploited (A. Hubert, R. Schafer, Magnetic Domains, Springer, 1998).

As a result of the reaction, the substrate on the surface changes the optical density and this can be detected with the mirage effect. In the mirage effect, the local warming of the surface by a focussed light beam can be measured on the basis of the consequent change in refractive index. Scanning the surface gives an image of the local absorption properties of the surface (A. Mandelis, Progress in Photothemial and Photoacoustic Science and Technology, Volume 1, Elsevier, New York 1992). A further thermal site-resolved procedure for the detection of the interaction reaction from the substrate is an array of microthermophiles, which measure the enthalpies of crystallisation or precipitation of the substrate (J. M. Köhler, M. Zieren, Thermochimica acta, 25, vol 310, 1998).

STM and AFM are also suitable for detecting the reaction with the substrate. In the atomic force microscope (AFM), a micro- or nano-tip scans the surfaces, which allows the surface topography to be measured (E. Braun, Y. Eichen, U. Sivan, G. Ben-Yoseph, Nature, 775, vol 391, 1998). The magnetic force microscope, MFM, uses a nanotip to detect local differences in magnetic susceptibility (A. Hubert, R. Schafer, Magnetic Domains, Springer, 1998). In scanning tunnelling microscopy STM, a nanotip is used to measure the tunnel current, in order to determine the surface topography at a nano level (O. Marti, M. Amrein, STM and SFM in biology, Academic Press Inc., San Diego, 1993)

More exotic procedures, such as ultrasound reflection tomography, can also be used. Tomographic procedures are procedures for preparing a 3-dimensional image on the basis of cross-sections (F. Natterer, Mathematische Methoden der Computer-Tomographie (Mathematical Methods of Computer Tomography), Westdt. Vlg., Wiesbaden, 1997). In ultrasound reflection tomography, the measurement of ultrasound tomography is used to produce the tomogram (V. Fleischer, F. Bergner, DGZfp NDT Conference Dresden 1997).

In a specific embodiment of the present invention, a method is made available which includes the following steps:

Detection of the time course of the formation of the precipitate on the array elements by taking pictures with a camera;

Conversion of the analog information contained in the images into a digital form;

Calculation of a virtual signal intensity for each array element on the basis of a curve function which describes the formation of the precipitate as a function of time;

Conversion of the virtual signal intensity into an artificial picture, which represents the virtual signal intensities of all array elements

In the context of the present invention, a picture means a group of pixels which depict the measured signal intensities for a probe array and which, for example, can be transferred directly to a screen or printer for recording.

In the context of the present invention, an artificial picture means a group of pixels which depict defined virtual signal intensities for a probe array and which, for example, can be transferred directly to a screen or printer for recording.

A further aspect of the present invention relates to a device for the performance of the procedure described above, in accordance with the invention. This includes:

a) an array substrate with probe array,

b) a reaction chamber,

c) a device for detecting a precipitate on an array element on which an interaction between target and probe has occurred, and

d) a computer which is programmed to:

collect the signal intensities recorded by the detection device;

the processing of the successively recorded signals, so as to guarantee that the time course of the precipitation on an array element is determined and that a virtual signal intensity is determined on the basis of the curve function which describes the formation of the precipitate as a function of time; and

if required, to guarantee the conversion of the virtual signal intensities into an analogue picture.

The detection device is preferably a camera, in particular a CCD or CMOS camera, or a similar camera, which usually records the whole area of the probe array.

As already mentioned above, time-resolved detection during the enhancement process through the deposition of the precipitate, as for example elementary silver on the gold particles acting as crystallisation seeds (nuclei), and the calculation of the relative degrees of occupancy from the time course, in the method in accordance with the invention, allow extreme increases in the dynamic resolution of the measured data, even if an 8 bit detection technique is used. The assembly of the device which is necessary for this is characterised by the mechanical inclusion of a reaction chamber and modified acquisition software. The software has the characteristic of allowing the processing of successive recordings. For this purpose, the grey values are determined for each element of the probe array for each time point. For all array elements, the virtual signal intensity is calculated in dependency on the time of precipitation. On the basis of this value for example, the grey values of the last measurement are related to the product of the rate and time of measurement, which then results in expansion of the range of measurement. In this way, excellent resolution between weak and intense probe/target interactions and exact quantification of the bound target is guaranteed, even if a cheap 8 bit camera is used.

In a preferred embodiment, the device in accordance with the invention includes a light source, which is preferably especially selected from the group of laser, light-emitting diode (LED) and a high pressure lamp.

The components of an exemplary assembly of a device in accordance with the invention for the optical detection of precipitation consist of a low power (500 mcd) light source, e.g. a LED, for homogenous illumination, and a detector, e.g. a CCD camera. Because of the enhancement effect from the catalytic deposition of the substrate, in particular when a gold/silver system is used, the changes in the optical properties of the system are so marked that a simple flat bed scanner, a diascanner or a similar instrument are adequate to detect the precipitation.

Typical detection times lie clearly under 1 second, whereas comparable sensitive CCD systems for the detection of fluorescence require about 10 to 80 seconds, so that cheap consumer cameras can be used, with signal transmission corresponding to the videonorm.

There is great scope for minituarising this system. The whole system can be planned as a self-standing hand instrument for field use. In addition, an especially preferred embodiment of the device in accordance with the invention is implementation as a highly integrated autonomous unit. This permits highly sensitive applications of microarrays, such as medical diagnosis, forensic medicine, bacterial screening, etc. These can be performed rapidly by laymen, independently of medical or biological laboratories.

In the following, the potential application of the method according to the present invention is described in tissue typing in transplantation medicine. The analysis of the structure, expression and inheritance of the immunologically relevant genes for transplantation and autoimmunity is of special interest, as there are highly polymorphic systems, both for specific antigen recognition (histocompatibility antigens, T-lymphocyte receptors) and for effector mechanisms (antibodies, Fc-receptors) and these are subject to highly complex genetic regulation mechanisms. Both weak and, particularly, strong transplantation antigens have a major effect on transplantation rejection. These strong antigens are called major histocompatibility antigens and are genetically coded within the major histocompatibility complex (MHC). The MHC has as yet only been detected in vertebrates and is called HLA (Human Leukocyte Antigen) in man. The HLA complex is located on the short arm of human chromosome 6 (6p21.1-6p21.3) and includes a section of about 3,500 kilobases. The HLA molecules may be classified very roughly into two classes (class I and class II), which are then split into further subgroups. The HLA gene products are responsible in their summation for the corresponding immunological properties of the organism. Their gene locations are inherited in very numerous allelic variations, of which the known number is increasing all the time. Allelic typing of the HLA system can be carried out exactly on an organism by serological and molecular biological analysis. Depending on the medical relevance of the cell species to be transplanted and the desired depth of the study, the number of allelic typings carried out can be varied. The deeper the typing and the better the subsequent agreement between the donor and recipient, the fewer are the problems which can be expected, such as tissue intolerance and rejection of the transplant. Aside from the various types of transplantation, unambiguous identification of individuals is also important in transfusion, disease associations and in forensic medicine.

The examples of embodiments include a proof of principle for the limit of detection and examples of expression monitoring. The proprietary principles used may however also be applied to other applications. Aside from quantitative analyses, such as the expression monitoring of organisms, numerous qualitative analyses may be performed.

For example, the HLA gene products are responsible in their summation for the corresponding immunological properties of the organism. Their gene locations are inherited in very numerous allelic variations, of which the known number is increasing all the time. Depending on the medical relevance of the cell species to be transplanted and the desired depth of the study, the number of allelic typings carried out can be varied. The deeper the typing and the better the subsequent agreement between the donor and recipient, the fewer are the problems which can be expected, such as tissue intolerance and rejection of the transplant. Since the discovery of MHC molecules, numerous procedures have been developed and used for characterising the polymorphism of these molecules and their genes. There is a fundamental difference between biochemical, cellular and serological techniques on the one hand and the techniques of molecular biology on the other. The former analyse exclusively the products of expression, for example by the use of specific antibodies, while the second group detects sequence differences in coding and non-coding sequences, for example by using the techniques of hybridisation and amplification of nucleic acids (Bidwell J., 1994 Advances in DNA-based HLA-typing methods. Immunol Today, 15(7):303-7). As a result of the described invention it would be possible, after isolation, appropriate labelling and possibly amplification to emphasise diagnostically relevant allelic structures in the sequence background of the individual genomic DNA, to carry out massive parallel hybridisation with a probe array (DNA chip), with the aim of carrying out HLA typing at a level as deep as possible. In comparison with other procedures, the detection of hybridisation described here and the signal enhancement, in combination with a simple detector, offers a highly economical procedure, with minimal time of diagnosis and maximal genomic typing, if known allele-specific probes are used.

A further area of application is in the area of pharmacology and diagnosis. In the metabolism of endogenous and exogenous substances (such as drugs) in the organism, a series of genetic polymorphisms, mutations, deletions, etc. and the associated functional effects at the protein level play an essential role. Individual genotypic distributions in these DNA sequences lead for example to phenotypic correlations with certain clinical pictures (for example, between the gene for p53 and mammary carcinoma and mitochondrial gene variations in the gene D loop, 16-S-rRNA, ND3-5, CytB, tRNATrp, tRNALeu and lung and bladder carcinoma (Fliss M S et. al (2000) Facile detection of mitochondrial DNA mutations in tumours and bodily fluids. Science. 17; 287(5460):2017-9.) or to different actions of xenobiotics on the organism. The latter has for example been demonstrated in detail with the cytochrome P 450 genes (CYP2D6, CYP2C19, CYP2A6, CYP2C9, CYP2E1), gluthathione-S-transferase genes (GSTM1, GSTT1), the N-acetyltransferase gene (NAT2), the apolipoprotein E gene (ApoE) and many others. The summary of the results of all these studies makes it possible to set up an array with DNA probes which detect the sequence differences in parallel. In the context of the enhanced detection of hybridisation as described above and the simple optical system, qualitative genotyping may be carried out rapidly and cheaply. This is relevant to both the individualised use of drugs, as a marker for the identification of individuals and to diagnosis.

The following examples and figures serve to explain the invention and should not be understood as to be limiting.

### EXAMPLES

### Example 1

### Detection of the Hybridisation of Nucleic Acids (Quantitative Analysis)

Preparation of the Carrier

An amino-modified 20-nucleotide with the sequence 5′-NH₂-CCTCTGCAGACTACTATTAC-3′ was covalently immobilised at a defined position on an epoxidated glass surface of 3×3 mm in area (“probe array”). For this purpose, 0.1 μl of a 5 μM solution of the oligonucleotide in 0.5 M phosphate buffer was overlaid on the glass surface and then dried at 37° C. The covalent binding of the overlaid oligonucleotides with the epoxide groups on the glass surface was established by baking the probe array for 30 min at 60° C. The probe array was then energetically washed with distilled water and then washed for 30 min in 100 mm KCl. After a further short wash in 100 mm KCl and then in distilled water, the probe array was dried for 10 min at 37° C.

Hybridisation of the Complementary Oligonucleotide

For the hybridisation, a complementary biotin-labelled 20 bp long oligonucleotide of sequence 5′-Bio-GTAATAGTAGTCTGCAGAGG-3′ was used. The reaction mixture was taken up in a total volume of 50 μl of buffer (0.25 M NaPO₄, 4.5% SDS, 1 mM EDTA in 1×SSC) in the following concentration steps: 10 nM, 1 nM, 100 pM, 10 pM, 1 pM, 100 fM, 10 fM, 1 fM.

A ready probe array was placed in the hybridisation mixture at each concentration step. The resulting hybridisation mixture was incubated for 5 min at 95° C. and then for 60 min at 50° C. After this, the probe array was shaken for 10 min each in 2×SSC+0.2% SDS, 2×SSC and 0.2×SSC (Maniatis et al., 1989), washed and blown dry with compressed air.

Detection of Hybridisation

A 1:50 dilution in 6×SSPE (52.5 g NaCl, 26.4 g NaH₂PO₄xH₂O, 2.22 g NaOH filled up to 1 volume with water) of a strepavidin-gold conjugate was applied to the probe array +0.005% Triton solution and incubated for 15 min at 30° C. The probe array was then washed with shaking for 10 min each in 2×SSC (17.5 g NaCl, 8.8 g Na citrate in 11H₂O, adjusted to pH 7.0 with 10 N NaOH)+0.2% SDS (sodium dodecylsulphate), 2×SSC and 0.2×SSC and blown dry in compressed air. Targets directly modified with gold particles were also used as an alternative to the gold conjugate of streptavidin.

The gold particles are now immobilised on the probe array. They were enhanced with 0.1% silver nitrate solution in 3% sodium carbonate and 0.02% formaldehyde solution. The mixture was prepared fresh shortly before the reaction. During the 15 min incubation, the reaction was monitored at 22° C. and under red light; it was continuously recorded with the device shown in FIG. 1.

The limit of detection was found to be <10 pM.

FIG. 3 shows the results of the hybridisation:

A—Hybridisation of the target when its concentration is 10 nM,

B—Hybridisation of the target when its concentration is 1 nM,

C—Hybridisation of the target when its concentration is 100 pM,

D—Hybridisation of the target when its concentration is 10 pM.

### Example 2

### Proof in Principle of the Use of the Procedure in Expression Profiling—Detection of the Hybridisation of Genomic RNA from Corynebacterium glutamicum against a probe array of 356 probes

DNA-Arrays are frequently used to measure the overall physiological state of cells (expression profiling). RNA is isolated from the corresponding cells for this purpose, labelled with a suitable method and hybridised in a probe array with complementary probes. In the following embodiment, the method in accordance with the invention is used to detect cellular RNAs from Corynebacterium glutamicum.

Preparation of the Probe Arrays

A probe array of 356 different amino-modified oligonucleotides of 25 or 30 bases in length and cDNAs of different lengths were used to prepare a probe array on a standardised and epoxidated microscope slide from the firm CLONDIAG Chip Technologies (Jena, Germany), which serves as the array substrate. All oligonucleotides were complementary to partial sequences of the aceA- and icd-genes. The probe arrays were prepared by arraying with the Micro-Grid I Arrayer of the firm Biorobotics Ltd. (Great Britain), in accordance with the instructions of the manufacturer, according to which the aminomodified DNAs were applied at a final concentration of 5 μM in 0.5 M phosphate buffer to the microscope slide and then dried. The covalent coupling between the applied oligonucleotides and the epoxide groups on the glass surface was formed by baking the microscope slide for 30 min at 60° C. The slides were then washed vigorously with distilled water and then washed for 30 min with 100 mM KCl. After a further short wash, first in 100 mM KCl and then in distilled water, the probe arrays were dried for 10 min at 37° C.

Preparation of Total RNA from Corynebacterium glutamicum

Total RNA from Corynebacterium glutamicum was isolated with the Fast RNA Kit (Bio 101 Ltd), according to the instructions of the manufacturer. 50 μg RNA was biotinylated with Biotin Chem Link (Boehringer Mannheim, Germany) at 85° C. for 30 min, according to the instructions of the manufacturer. The RNA was then concentrated on Microcon-30 columns (Millipore Ltd), in accordance with the instructions of the manufacturer and then washed several times with deionised and RNAse free water. The eluate was then concentrated under vacuum to 5 μl.

Hybridisation of the RNA

The biotinylated RNA was taken up in 100 μl hybridisation buffer (0.25 M NaPO₄, 4.5% SDS, 1 mM EDTA in 1×SSC) and denatured for 3 min at 65° C. The DNA-coated surface of the slide was covered with a hybridisation chamber (Hybrislip, Sigma, Deisenhofen, Germany). The slide was then brought to 50° C. on a thermostatted shaker with an insert for microtitre plates (Eppendorf, Hamburg, Germany). The chamber was then filled with the denatured hybridisation solution and the hybridisation chamber closed in accordance with the instructions of the manufacturer. The incubation was continued for 60 min at 50° C. The hybridisation solution was then taken off and the hybridisation chamber removed. The slides were then washed with shaking for 10 min at 30° C. in 2×SSC+0.2% SDS and for 10 min each at room temperature in 2×SSC and 0.2×SSC (Maniatis et al., 1989) and blown dry with compressed air.

Detection of Hybridisation

A 1:50 dilution in 6×SSPE+0.005% Triton (Maniatis et al., 1989) of a streptavidin-gold conjugate (EM.STP5, British BioCell International Ltd) was applied to the slide, which was then incubated for 15 min at 30° C. The probe arrays were then washed and shaken for 10 min each in 2×SSC+0.2% SDS, 2×SSC and 0.2×SSC and dried in compressed air.

The immobilised gold particles on the probe array were enhanced with the LM/EM Silver Enhancing Kit (SEKL15, British BioCell International). In accordance with the instructions of the manufacturer, 2 drops each of the initiator and enhancer solutions were mixed and 15 μl thereof pipetted onto the surface of the probe array. During the 15 min incubation period, the reaction was monitored 22° C. under red light and the reaction recorded continuously with the device which is depicted in FIG. 1. A final evaluation of the changes can also be carried out after 15 min incubation (see FIG. 4).

### Example 3

### Detection and Specificity of the Hybridisation of Nucleic Acids

Preparation of the Probe Array

16 amino-modified oligonucleotides (probes) with a length of 16 nucleotides each were applied at defined sites with a MicroGrid II Arrayer (BioRobotics Ltd) and covalently immobilised (array elements) on an epoxidated 3D microscope slide (75 mm×25 mm) with a glass surface (Elipsa Ltd). The sequences of the oligonucleotides were as follows (each with a 3′-NH₂-modification):

```
  1: 3′- ATG GCG TTT AGA ACC C -5′
  2: 3′- ATG CCG TAT GGA ATC C -5′
  3: 3′- ATG TCG TGT CGA AAC C -5′
  4: 3′- ATG ACG TCT TGA AGC C -5′
  5: 3′- ACG GCA TTT AGT ACC G -5′
  6: 3′- ACG CCA TAT GGT ATC G -5′
  7: 3′- ACG TCA TGT CGT AAC G -5′
  8: 3′- ACG ACA TCT TGT AGC G -5′
  9: 3′- AGG GCT TTT AGC ACC A -5′
10: 3′- AGG CCT TAT GGC ATC A -5′
11: 3′- AGG TCT TGT CGC AAC A -5′
12: 3′- AGG ACT TCT TGC AGC A -5′
13: 3′- AAG GCC TTT AGG ACC T -5′
14: 3′- AAG CCC TAT GGG ATC T -5′
15: 3′- AAG ACC TCT TGG AGC T -5′
16: 3′- AAG TCC TGT CGG AAC T -5′
```

A single complete (quadratic) probe array on the surface of the slide consisted of 10×10=100 applied probes in all. Each of the oligonucleotide probes was applied at least 5 times on the probe array (for the array composition see FIG. 5). The probes were 0.2 mm apart and the whole probe array covered an area of 2 mm×2 mm. In this way, more than 100 identical probe arrays could be produced for each slide.

The probes were applied as 10 μM solutions of each oligonucleotide in 0.1 M phosphate buffer/5%-sodium sulphate. After application and drying, the probes were coupled to the epoxide groups on the glass surface by being baked for 30 min at 60° C. The slides were then washed and blocked in the following sequence:

5 min in 600 ml double distilled H₂O+600 μl Triton ×100

2×2 min in 600 ml double distilled H₂O+60 μl HCl (conc.)

30 min in 100 mM KCl solution

Wash 1 min in double distilled H₂O

Incubate for 15 min at 50° C. in a glass dish in 75 ml double distilled H₂O+25 ml ethylene glycol +20 μl HCl (conc.).

Wash 1 min in double distilled H₂O

Dry in compressed air.

After washing and drying, the slides were cut up into pieces (called “chips” below), which were 3.25 mm×3.25 mm in size. On each of these chips there was exactly one probe array, which was 2 mm×2 mm in size.

Hybridisation of the Probe Arrays

The complementary biotin-labelled 16 bp long oligonucleotides were available as targets for the hybridisation of each of the 16 oligonucleotide probes in the probe array.

The complementary target “9b” for oligonculeotide probe 9 has the following sequence and is given here as the only example:

5′-Biotin TCC CGA AAA TCG TGG T-3′

The hybridisation reaction was carried out in 6×SSPE buffer (52.59 g NaCl, 8.28 g NaH₂PO₄×H₂O, 2.22 g EDTA×2H₂O in 1 l double distilled H₂O, adjusted to pH 7.4 with NaOH)/0.1% SDS in a total volume of 70 μl with target concentration steps of 100 nM, 10 nM, 1 nM, 100 pM, 10 pM and 1 pM. For each concentration step, a chip with the probe array was added to the hybridisation solution, heated for 5 min at 95° C. and then incubated with shaking for 60 min at 30° C. The chip was then transferred into a new reaction vessel with 500 μl hybridisation buffer (without target) and washed with shaking for 10 min at 55° C. or 60° C. The chips were then washed with shaking for further periods of 10 min in 2×SSC/0.2% SDS (500 μl at 30° C.), 2×SSC (500 μl at 20° C.) and 0.2×SSC (500 μl at 20° C.) and dried (Eppendorf Concentrator).

Detection of Hybridisation (Conjugation and Silver Staining)

The hybridised and dried chips were transferred to a new reaction vessel with μl of a streptavidin-gold conjugate solution in 6×SSPE/0.1% SDS buffer and incubated there for 15 min at 30° C. 5 nm gold particles were used for the streptavidin-gold conjugate (British Biocell International, EM.STP5). The conjugate was present in the solution at a concentration of 500 pg Streptavidin/μl.

After the conjugation step, the chips were washed with shaking for 10 min each in 2×SSC/0.2% SDS (500 μl at 30° C.), 2×SSC (500 μl at 20° C.) and 0.2×SSC (500 μl at 20° C.) and then dried (Eppendorf Concentrator).

As an alternative to this procedure, the streptavidin-gold conjugate coupling was performed directly in the hybridisation solution. For this purpose, the streptavidin-gold conjugate was added directly to the hybridisation solution after the 60 min hybridisation and then incubated for a further 15 min at 30° C. After this, the chip was transferred to a new reaction vessel with 500 μl hybridisation buffer (without target) and washed with shaking for 10 min at 55° C. or 60° C. After this, the chips were washed with shaking for 10 min each in 2×SSC/0.2% SDS (500 μl at 30° C.), 2×SSC (500 μl at 20° C.) and 0.2×SSC (500 μl at 20° C.) and then dried (Eppendorf Concentrator).

For the silver enhancement, the chips were transferred to a new reaction vessel and incubated with shaking for 10 min at 25° C. in ca. 100 μl of a silver enhancing solution (British Biocell International, SEKL15). The incubation solution was produced from one drop each of initiator and enhancer solution. The chip was then washed for 2 min in 500 μl 0.2×SSC and dried (Eppendorf Concentrator).

Two examples of the hybridisation and its detection are shown in FIGS. 6a and 6b (transmission photos).

### Example 4

### Detection and Specificity of the Hybridisation of Nucleic Acids

More than 800 mutations of the CFTR gene have been described in the literature which can lead to the symptoms of cystic fibrosis. There are three types of mutation in the CFTR gene:

Base exchange (here: point mutations)

Insertions

Deletions

For all three types of mutation, it is to be tested whether the wild type (pm) can be distinguished from the mutation (mm) with silver enhancement detection. The probes and targets were prepared by Ogham Ltd (Münster, Germany).

Preparation of the Probe Arrays

10 aminomodified oligonucleotides (probes) with a length of 16 to 22 nucleotides were applied to defined sites on the glass surface of an epoxidated 3D microscope slide (75 mm×25 mm) (Elipsa Ltd) with a MicroGrid II Arrayer (BioRobotics Ltd) and covalently immobilised an (array elements). The 10 probes are divided into 5 pairs, where the first is always the wild type and the second the mutation. The probe pair 1 and 2 is a point mutation, the pair 3 and 4 a deletion and the pairs 5/6, 7/8 and 9/10 insertions. The sequence of the oligonucleotides was as follows:

Sequence in the 5′-3′ direction with 3′-NH₂ modification:

| | | |
|---|---|---|
| 1: | GATCTTCGCCTTACTG | pm |
| 2: | GATCTTCACCTTACTG | mm |
| 3: | GAAACACCAAAGATGATA | pm |
| 4: | GAAACACC GATGATA | mm |
| 5: | CTTCTAATTA TTTGGTATGT | pm |
| 6: | CTTCTAATTATTTTGGTATGT | mm |
| 7: | GAGTTCTTCTAATTA TTTGG | pm |
| 8: | GAGTTCTTCTAATTATTTTGG | mm |
| 9: | TTTTAGAGTTCTTCTAATTA T | pm |
| 10: | TTTTAGAGTTCTTCTAATTATT | mm |

Probe pair 3 (wild type) and 4 (deletion) contains the most frequent mutation (70% of all cases) which codes for cystic fibrosis.

A single complete (quadratic) probe array on the surface of the microscope slide consisted in all of 10×10=100 applied probes. Each of the 10 oligonucleotide probes was applied 8 to 10 times on the probe array (for the structure of the array see FIG. 7). The distance between the probes was 0.2 mm and the total probe array covered an area of 2 mm×2 mm. In this way, more than 100 identical probe arrays could be produced on each slide.

The probes were applied from 10 μM of each oligonuculeotide in 0.1 M phosphate buffer/5% sodium sulphate. After application and drying, the probes were covalently coupled to the epoxide groups on the glass surface by 30 min baking at 60° C. The slides were then washed and blocked in the following sequence:

5 min in 600 ml double distilled H₂O+600 μl Triton ×100

2×2 min in 600 ml double distilled H₂O+60 μl HCl (conc.)

30 min in 100 mM KCl solution

Wash for 1 min in double distilled H₂O

Incubate for 15 min at 50° C. in a glass dish in 75 ml double distilled H₂O+25 ml

ethylene glycol +20 μl HCl (conc.).

Wash for 1 min in double distilled H₂O.

Dry in compressed air.

After washing and drying, the slides were cut up into pieces (called “chips” below), which were 3.25 mm×3.25 mm in size. On each of these chips there was exactly one probe array, which was 2 mm×2 mm in size.

Hybridisation and Conjugation of the Probe Array

3 complementary biotin-labelled targets were available for hybridisation to the perfect match (pm) 10 oligonucleotide probes. Target 1 covered probe pair 1 and 2, target 2 pair 3 and 4 and target 3 probe pairs 5/6, 7/8 and 9/10. The sequences of the targets were:

Target 1:
5′-Biotin- CTCAGTAAGGCGAAGATCTT-3′
Target 2:
5′-Biotin- AATATCATCTTTGGTGTTTCCT-3′
Target 3:
5′-Biotin- GAACATACCAAATAATTAGAAGAACTCTAAAACA-3′

The hybridisation reaction was performed in 6×SSPE-Puffer (52.59 g NaCl, 8.28 g NaH₂PO₄×H₂O, 2.22 g EDTA×2H₂O in 11 double distilled H₂O, adjusted to pH 7.4 with NaOH)/0.1% SDS, in an overall volume of 70 μl with different target concentration steps. For this purpose, the chip with the probe array was placed in the hybridisation solution, heated for 5 min at 95° C., and then incubated with shaking for 60 min at 30° C.

After the 60 min hybridisation, a streptavidin gold conjugate was added directly to the hybridisation solution and then incubated for a further 15 min at 30° C. 5 nm gold particles (British Biocell International, EM.STP5) were used for the streptavidin gold conjugate. The concentration of the conjugate in the experiment was 500 pg streptavidin/μl.

After hybridisation and conjugation, the chip was transferred to a new reaction vessel with 500 μl hybridisation buffer (without target) and washed with shaking for 10 min at 55° C. The chips were then washed with shaking for 10 min each in 2×SSC/0.2% SDS (500 μl at 30° C.), 2×SSC (500 μl at 20° C.) and 0.2×SSC (500 μl at 20° C.) and then dried (Eppendorf Concentrator).

Silver Enhancement

For silver enhancement, the chips were transferred to a new reaction vessel and incubated with shaking for 10 min at 25° C. in ca. 100 μl of a silver enhancement solution (British Biocell International, SEKL15). The incubation solution was produced by mixing one drop each of initiator and enhancement solutions. The chip was then washed for 2 min in 500 μl 0.2×SSC and dried (Eppendorf Concentrator).

The results of the 3 hybridisations and their detection are shown in FIGS. 8, 9 and 10 (transmission images). Although the point mutation (FIG. 8) and the deletion (FIG. 9) allow clear distinction between wild type (pm) and mutation (mm), this does not apply to the insertion (FIG. 10). In this case, the mutation (probe 10) even gives a stronger signal than the wild type (probe 9). In this experimental design, the limit of detection for hybridisation lies at a target concentration of 10 pM.

### Example 5

### Proof of Principle for the Use of the Procedure with an Oligonucleotide Gold Conjugate

Preparation of the Probe Array

16 amino-modified oligonucleotides (probes) with a length of 16 nucleotides each were applied at defined sites and covalently immobilised (array elements) to an epoxidated glass surface of a 3D microscope slide (75 mm×25 mm) (Elipsa Ltd), using a MicroGrid II Arrayer (BioRobotics Ltd). The oligonucleotides each had a 3′ modification; their sequences were as follows:

```
  1: 3′- ATG GCG TTT AGA ACC C -5′
  2: 3′- ATG CCG TAT GGA ATC C -5′
  3: 3′- ATG TCG TGT CGA AAC C -5′
  4: 3′- ATG ACG TCT TGA AGC C -5′
  5: 3′- ACG GCA TTT AGT ACC G -5′
  6: 3′- ACG CCA TAT GGT ATC G -5′
  7: 3′- ACG TCA TGT CGT AAC G -5′
  8: 3′- ACG ACA TCT TGT AGC G -5′
  9: 3′- AGG GCT TTT AGC ACC A -5′
10: 3′- AGG CCT TAT GGC ATC A -5′
11: 3′- AGG TCT TGT CGC AAC A -5′
12: 3′- AGG ACT TCT TGC AGC A -5′
13: 3′- AAG GCC TTT AGG ACC T -5′
14: 3′- AAG CCC TAT GGG ATC T -5′
15: 3′- AAG ACC TCT TGG AGC T -5′
16: 3′- AAG TCC TGT CGG AAC T -5′
```

A single complete (quadratic) probe array on the surface of the slide consisted of 10×10=100 applied probes in all. Each of the 16 oligonucleotide probes was applied at least 5 times on the probe array (for the array composition see FIG. 11). The probes were 0.2 mm apart and the whole probe array covered an area of 2 mm×2 mm. In this way, more than 100 identical probe arrays could be produced for each slide.

The probes were applied as 10 μM solution of each oligonucleotide in 0.1 M phosphate buffer/5%-sodium sulphate. After application and drying, the probes were coupled to the epoxide groups on the glass surface by being baked for 30 min at 60° C. The slides were then washed and blocked in the following sequence:

5 min in 600 ml double distilled H₂O+600 μl Triton ×100

2×2 min in 600 ml double distilled H₂O+60 μl HCl (conc.)

30 min in 100 mM KCl solution

Wash 1 min in double distilled H₂O

Incubate for 15 min at 50° C. in a glass dish in 75 ml double distilled H₂O+25 ml

ethylene glycol +20 μl HCl (conc.).

Wash 1 min in double distilled H₂O

Dry in compressed air.

After washing and drying, the slides were cut up into pieces (called “chips” below), which were 3.25 mm×3.25 mm in size. On each of these chips there was exactly one probe array, which was 2 mm×2 mm in size.

Preparation of the Oligonucleotide-Gold Conjugate

To prepare the oligonucleotide-gold conjugate, 5.4 mmol of a modified oligonucleotide (dissolved in 80 μl double distilled H₂O) with the sequence 5′-thiol-TTTTTTTTTTTTTTTTTTT-3′ (“T20-thiol”) were mixed with 6 mmol monomaleimido-nanogold (Nanoprobes Ltd) and incubated for 24 h at 4° C. The nanogold was dissolved in 20 μl isopropanol and 180 μl double distilled H₂O.

Hybridisation and Conjugation of the Probe Array

The complementary 36 hp oligonucleotides were available as targets for all 16 oligonucleotide probes in the probe array. These targets were modified with a 3′-polyA tail. One example of this is the target “9c”, which is complementary to oligonucleotide probe 9. This has the following sequence:

5′-TCCCGAAAATCGTGGTAAAAAAAAAAAAAAAAAAAA-3′

The hybridisation reaction was performed in 6×SSPE buffer (52.59 g NaCl, 8.28 g NaH₂PO₄×H₂O, 2.22 g EDTA×2H₂O in 1 l double distilled H₂O, adjusted to pH pH 7.4 with NaOH)/0.1% SDS, in an overall volume of 70 μl with stepped target concentrations. At each concentration step, the chip with the probe array was added to the hybridisation step, heated for 5 min at 95° C. and then incubated with shaking for 60 min at 30° C. After this, different dilutions of the T20-nanogold conjugate were added to the hybridisation solution and incubated for a further 30 min at 30° C.

After this, the chip was transferred to a new reaction vessel with 500 μl hybridisation buffer (without target and T20-nanogold) and washed with shaking for 10 min at 55° C. or 60° C. Finally, the chips were washed with shaking for 10 min each in 2×SSC/0.2% SDS (500 μl at 30° C.), 2×SSC (500 μl at 20° C.) and 0.2×SSC (500 μl at 20° C.) and then dried (Eppendorf Concentrator).

Silver Enhancement

For silver enhancement, the chips were transferred into a new reaction vessel and incubated with shaking for 10 min at 25° C. in ca. 100 μl of a silver development solution (British Biocell International, SEKL15). The incubation solution was prepared by mixing one drop each of initiator and enhancement solutions. The chip was then washed for 2 min in 500 μl 0.2×SSC and dried (Eppendorf Concentrator).

### Example 6

### Detection and Specificity of the Hybridisation of Nucleic Acids

More than 800 mutations are known in the literature which can lead to the clinical appearance of cystic fibrosis. Three types of mutation occur in the CFTR gene:

Base exchange (here: point mutations)

Insertions

Deletions

Tests are to be carried out for all three types of mutation, to establish whether the wild types (pm) can be distinguished from the mutations (mm) by the silver enhancement detection.

Probes and targets were prepared by Ogham Ltd.

Preparation of the Probe Arrays

10 aminomodified oligonucleotides (probes) with a length of 16 to 22 nucleotides were applied to defined sites on the glass surface of an epoxidated 3D microscope slide (75 mm×25 mm) (Elipsa Ltd) with a MicroGrid II Arrayer (BioRobotics Ltd) and covalently immobilised an (array elements). The 10 probes are divided into 5 pairs, where the first is always the wild type and the second the mutation. The probe pair 1 and 2 is a point mutation, the pair 3 and 4 a deletion and the pairs 5/6, 7/8 and 9/10 insertions. The sequence of the oligonucleotides was as follows:

Sequence in the 5′-3′ direction with 3′-NH₂ modification:

| | | |
|---|---|---|
| 1: | GATCTTCGCCTTACTG | pm |
| 2: | GATCTTCACCTTACTG | mm |
| 3: | GAAACACCAAAGATGATA | pm |
| 4: | GAAACACC GATGATA | mm |
| 5: | CTTCTAATTA TTTGGTATGT | pm |
| 6: | CTTCTAATTATTTTGGTATGT | mm |
| 7: | GAGTTCTTCTAATTA TTTGG | pm |
| 8: | GAGTTCTTCTAATTATTTTGG | mm |
| 9: | TTTTAGAGTTCTTCTAATTA T | pm |
| 10: | TTTTAGAGTTCTTCTAATTATT | mm |

The probe pair 3 (wild type) and 4 (deletion) corresponds to the most frequent mutation which codes for cystic fibrosis (70% of all cases).

A single complete (quadratic) probe array on the surface of the slide consisted of 10×10=100 applied probes in all. Each of the oligonucleotide probes was applied 8 to 10 times on the probe array (for the array composition see FIG. 13). The probes were 0.2 mm apart and the whole probe array covered an area of 2 mm×2 mm. In this way, more than 100 identical probe arrays could be produced for each slide.

The probes were applied as 10 μM solution of each oligonucleotide in 0.1 M phosphate buffer/5%-sodium sulphate. After application and drying, the probes were coupled to the epoxide groups on the glass surface by being baked for 30 min at 60° C. The slides were then washed and blocked in the following sequence:

5 min in 600 ml double distilled H₂O+600 μl Triton ×100

2×2 min in 600 ml double distilled H₂O+60 μl HCl (conc.)

30 min in 100 mM KCl solution

Rinse 1 min in double distilled H₂O

Incubate for 15 min at 50° C. in a glass dish in 75 ml double distilled H₂O+25 ml

ethylene glycol +20 μl HCl (conc.).

Rinse 1 min in double distilled H₂O

Dry in compressed air.

After washing and drying the slides, they were cut up into pieces (called “chips” below), which were 3.25 mm×3.25 mm in size. On each of these chips there was exactly one probe array, which was 2 mm×2 mm in size.

Hybridisation and Conjugation of the Probe Arrays

3 complementary biotin-labelled targets were available for hybridisation to the perfect match (pm) 10 oligonucleotide probes. Target 1 covered probe pair 1 and 2, target 2 pair 3 and 4 and target 3 probe pairs 5/6, 7/8 and 9/10. The sequences of the targets were:

Target 1:
5′-Biotin- CTCAGTAAGGCGAAGATCTT-3′
Target 2:
5′-Biotin- AATATCATCTTTGGTGTTTCCT-3′
Target 3:
5′-Biotin- GAACATACCAAATAATTAGAAGAACTCTAAAACA-3′

The hybridisation reaction was performed in 6×SSPE-Puffer (52.59 g NaCl, 8.28 g NaH₂PO₄×H₂O, 2.22 g EDTA×2H₂O in 1 l double distilled H₂O, adjusted to pH 7.4 with NaOH)/0.1% SDS in a total volume of 70 μl, with all three targets being added at concentrations of 100 pM. For this purpose, a chip with the probe array was added to the hybridisation solution, heated for 5 min at 95° C., then incubated with shaking for 60 min at 30° C.

After 60 min hybridisation, the streptavidin-gold conjugate was added directly to the hybridisation solution and then incubated for a further 15 min at 30° C. 5 nm gold particles were used for the streptavidin-gold conjugate (British Biocell International, EM.STP5). The conjugate was used in the experiment at a concentration of 500 pg Streptavidin/μl.

After the hybridisation and conjugation, the chip was transferred to a new reaction vessel with 500 μl hybridisation buffer (without target) and washed with shaking for 110 min at 55° C. The chips were then washed for 10 min each in 2×SSC/0.2% SDS (500 μl at 30° C.), 2×SSC (500 μl at 20° C.) and 0.2×SSC (500 μl at 20° C.) and then dried (Eppendorf Concentrator).

Silver Enhancement, Detection and Evaluation

For the silver enhancement, the chips were fixed in a closed reaction chamber (see FIG. 1) and overlaid with a silver enhancement solution (British Biocell International, SEKL15). The incubation solution was prepared by mixing one drop each of initiator and enhancer solutions. During the 30 min incubation at 21° C., the time course of the silver enhancement was documented with one photo per min (a red LED was the light source for this).

The pictures were then evaluated with the picture evaluation software IconoClust (Clondiag Ltd).

As an example, FIGS. 14a and 14b show the chip photographs 5 and 10 min after the start of the silver enhancement. The hybridisation was carried out with target 2. FIG. 15 shows the time course of this reaction.

### Example 7

### Detection and Specificity of the Hybridisation of Nucleic Acids—Measurement of Time Courses

More than 800 mutations of the CFTR gene have been described in the literature which can lead to the symptoms of cystic fibrosis. There are three types of mutation in the CFTR gene:

Base exchange (here: point mutations)

Insertions

Deletions

For all three types of mutation, it is to be tested whether the wild type (pm) can be distinguished from the mutation (mm) with silver enhancement detection.

The probes and targets were provided by Ogham Ltd (Münster, Germany).

Preparation of the Probe Arrays

10 amino-modified oligonucleotides (probes) with a length of 16 to 22 nucleotides were applied to defined sites on the glass surface of an epoxidated 3D microscope slide (75 mm×25 mm) (Elipsa Ltd) with a MicroGrid II Arrayer (BioRobotics Ltd) and covalently immobilised an (array elements). The 10 probes are divided into 5 pairs, where the first is always the wild type and the second the mutation. The probe pair 1 and 2 is a point mutation, the pair 3 and 4 a deletion and the pairs 5/6, 7/8 and 9/10 insertions. The sequence of the oligonucleotides was as follows:

Sequence in the 5′-3′ direction with 3′-NH₂ modification:

| | | |
|---|---|---|
| 1: | GATCTTCGCCTTACTG | pm |
| 2: | GATCTTCACCTTACTG | mm |
| 3: | GAAACACCAAAGATGATA | pm |
| 4: | GAAACACC GATGATA | mm |
| 5: | CTTCTAATTA TTTGGTATGT | pm |
| 6: | CTTCTAATTATTTTGGTATGT | mm |
| 7: | GAGTTCTTCTAATTA TTTGG | pm |
| 8: | GAGTTCTTCTAATTATTTTGG | mm |
| 9: | TTTTAGAGTTCTTCTAATTAT | pm |
| 10: | TTTTAGAGTTCTTCTAATTATT | mm |

Probe pair 3 (wild type) and 4 (deletion) contains the most frequent mutation (70% of all cases) which codes for cystic fibrosis.

A single complete (quadratic) probe array on the surface of the microscope slide consisted in all of 10×10=100 applied probes. Each of the 10 oligonucleotide probes was applied 8 to 10 times on the probe array (for the structure of the array see FIG. 16). The distance between the probes was 0.2 mm and the total probe array covered an area of 2 mm×2 mm. In this way, more than 100 identical probe arrays could be produced on each slide.

The probes were applied from 10 μM of each oligonuculeotide in 0.1 M phosphate buffer/5% sodium sulphate. After application and drying, the probes were covalently coupled to the epoxide groups on the glass surface by 30 min baking at 60° C. The slides were then washed and blocked in the following sequence:

5 min in 600 ml double distilled H₂O+600 μl Triton ×100

2×2 min in 600 ml double distilled H₂O+60 μl HCl (conc.)

30 min in 100 mM KCl solution

Wash for 1 min in double distilled H₂O

Incubate for 15 min at 50° C. in a glass dish in 75 ml double distilled H₂O+25 ml

ethylene glycol +20 μl HCl (conc.).

Wash for 1 min in double distilled H₂O.

Dry in compressed air.

After washing and drying, the slides were cut up into pieces (called “chips” below), which were 3.25 mm×3.25 mm in size. On each of these chips there was exactly one probe array, which was 2 mm×2 mm in size.

Hybridisation and Conjugation of the Probe Arrays

3 complementary biotin-labelled targets were available for hybridisation to the perfect match (pm) 10 oligonucleotide probes. Target 1 covered probe pair 1 and 2, target 2 pair 3 and 4 and target 3 probe pairs 5/6, 7/8 and 9/10. The sequences of the targets were:

Target 1:
5′-Biotin- CTCAGTAAGGCGAAGATCTT-3′
Target 2:
5′-Biotin- AATATCATCTTTGGTGTTTCCT-3′
Target 3:
5′-Biotin- GAACATACCAAATAATTAGAAGAACTCTAAAACA-3′

The hybridisation reaction was performed in 6×SSPE-Puffer (52.59 g NaCl, 8.28 g NaH₂PO₄×H₂O, 2.22 g EDTA×2H₂O in 11 double distilled H₂O, adjusted to pH 7.4 with NaOH)/0.1% SDS in a total volume of 70 μl, with all three targets being added at concentrations of 100 pM. For this purpose, a chip with the probe array was added to the hybridisation solution, heated for 5 min at 95° C., then incubated with shaking for 60 min at 30° C.

After 60 min hybridisation, the streptavidin-gold conjugate was added directly to the hybridisation solution and then incubated for a further 15 min at 30° C. 5 nm gold particles were used for the streptavidin-gold conjugate (British Biocell International, EM.STP5). The conjugate was used in the experiment at a concentration of 125 pg Streptavidin/μl.

After the hybridisation and conjugation, the chip was transferred to a new reaction vessel with 500 μl hybridisation buffer (without target) and washed with shaking for 10 min at 55° C. The chips were then washed for 10 min each in 2×SSC/0.2% SDS (500 μl at 30° C.), 2×SSC (500 μl at 20° C.) and 0.2×SSC (500 μl at 20° C.) and then dried (Eppendorf Concentrator).

Silver Enhancement, Detection and Evaluation

For the silver enhancement, the chips were fixed in a closed reaction chamber (see FIG. 1) and overlaid with a silver enhancement solution (British Biocell International, SEKL15). The incubation solution was prepared by mixing one drop each of initiator and enhancer solutions. During the 20 min incubation at 27° C., the time course of the silver enhancement was documented with one photo per 10 sec (a red LED was the light source for this). The pictures were then evaluated with the picture evaluation software IconoClust (Clondiag Ltd).

The results are shown in FIGS. 17 to 20 and in Table 2. The linear regression lines for each probe were determined in the range of exponential increase of each curve and are typical for each target concentration. On this basis, an unknown target concentration can be estimated. The condition for this is that the same quantity of conjugate is used, the same concentration of immobilised probe and the same experimental parameters.

Table 2: Linear regression equations for selected probes (array elements) and the chip background. The rise in each regression line is printed bold (x: time in min since the start of the silver enhancement, y: signal intensity in the valid min range, hybridisation with target 3 at concentrations 100 nM and 1 nM).

| Element | | | | | |
|---|---|---|---|---|---|
| of the Probe | Target | | | | |
| Array | Concentration | Time Range | Equation f(x) | R² | Standard Error |
| Background | 100 nM | 1-20 | y = 0.0551 + (0.013*x) | 0.971 | 0.014 |
| Background | 1 nM | 1-20 | y = 0.0161 + (0.013*x) | 0.984 | 0.01 |
| Probe 5 pm | 100 nM | 4-13 | y = −0.263 + (0.0740*x) | 0.993 | 0.021 |
| Probe 5 pm | 1 nM | 4-13 | y = −0.259 + (0.0677*x) | 0.989 | 0.023 |
| Probe 6 mm | 100 nM | 4-13 | y = −0.246 + (0.0647*x) | 0.991 | 0.02 |
| Probe 6 mm | 1 nM | 4-13 | y = −0.153 + (0.0417*x) | 0.969 | 0.024 |

### FIGURES

FIG. 1: Device for the qualitative and/or qualitative detection of interactions between probes and targets

FIG. 2: Record of the time course of the hybridisation results shown in FIG. 3.

FIG. 3: Depiction of the hybridisation results

A—Hybridisation of the target at a concentration of 10 nM

B—Hybridisation of the target at a concentration of 1 nM

C—Hybridisation of the target at a concentration of 100 pM

D—Hybridisation of the target at a concentration of 10 pM

FIG. 4: Detection of the hybridisation of genomic RNA from Corynebacterium glutamicum with a probe array of 356 probes—pattern resulting after 15 min incubation

FIG. 5: Assembly of an array which is 2 mm×2 mm in size and contains 10×10=100 probes.

The numbers 1-16 each stand for an oligonucleotide probe which has been applied 5 or 6 times to the array; “M” stands for a mixture of markers, which includes an immobilised biotin-labelled oligonucleotide; 1 Position on+in the array is not occupied.

FIG. 6: Probe array after hybridisation, conjugation and silver enhancement (for array assembly cf. FIG. 5).

a) left figure: target 9b (100 nM) hybridisation at 30° C.; First washing step at 60° C.; streptavidin gold conjugate (500 pg/μl); silver enhancement: 10 min at 25° C.

Aside from specific probe 9 (and the markers), a weak non-specific signal from probe 13 is recognisable.

b) right figure: target 9b (100 pM) hybridisation at 30° C., followed by direct addition of streptavidin gold conjugate (500 pg/μl); 1. washing step at 60° C., silver enhancement: 10 min at 25° C.

FIG. 7: Assembly of an array which is 2 mm×2 mm in size and contains 10×10=100 probes.

The numbers 1-10 each stand for an oligonucleotide probe which has been applied 8 to 10 times to the array; “M” stands for a mixture of markers, which includes an immobilised biotin-labelled oligonucleotide.

FIG. 8: Probe array after hybridisation, conjugation and silver enhancement (for array assembly cf. FIG. 7)

Target 1 (100 pM) hybridisation at 30° C., followed by direct addition of streptavidin-gold conjugate (500 pg/μl); 1. Washing step at 55° C., silver enhancement: 10 min at 25° C.

FIG. 9: Probe array after hybridisation, conjugation and silver enhancement (for array assembly cf. FIG. 1)

Target 2 (100 pM) hybridisation at 30° C., followed by direct addition of streptavidin-gold conjugate (500 pg/μl); 1. Washing step at 55° C., silver enhancement: 10 min at 25° C.

FIG. 10: Probe array after hybridisation, conjugation and silver enhancement (for array assembly cf. FIG. 1)

Target 3 (100 pM) hybridisation at 30° C., followed by direct addition of streptavidin-gold conjugate (500 pg/μl); 1. Washing step at 55° C., silver enhancement: 10 min at 25° C.

FIG. 11: Assembly of an array which is 2 mm×2 mm in size and contains 10×10=100 probes.

The numbers 1-16 each stand for an oligonucleotide probe which has been applied 5 or 6 times to the array; “M” stands for a mixture of markers, which includes an immobilised biotin-labelled oligonucleotide. 1 position on the array is not occupied.

FIG. 12: Probe array after hybridisation, conjugation and silver enhancement (for array assembly cf. FIG. 11)

Target 9c (1 nM) 60 min hybridisation at 30° C., followed by addition of T20 nanogold (1:100 dilution) and further incubation for 30 min at 30° C.; 1. Washing step at 55° C., silver enhancement: 10 min at 25° C.

Apart from the strong specific signal with probe 9 (and the markers), the other probes give a weak signal; some of the spots are smeared and inhomogenous. This was caused by impurities in the array when the probes were being applied.

FIG. 13: Assembly of an array which is 2 mm×2 mm in size and contains 10×10=100 probes.

The numbers 1-10 each stand for an oligonucleotide probe which has been applied 5 or 6 times to the array; “M” stands for a mixture of markers, which includes an immobilised biotin-labelled oligonucleotide.

FIG. 14: Probe array after hybridisation, conjugation and silver enhancement (for array assembly cf. FIG. 1)

Target 2 (100 pM) hybridisation at 30° C. followed by direct addition of streptavidin-gold conjugate (500 pg/μl); 1. Washing step at 55° C.

a) left picture: 5 min after the start of the silver enhancement

b) right picture: 10 min after the start of the silver enhancement

FIG. 15: Time course of the silver enhancement (cf. FIGS. 13 and 14)

Measurement every min; each point of measurement is the mean of 10 repeated spots

pm: Perfect match probe (probe no. 3)

mm: Mismatch probe (probe no. 4)

Target 2 (100 pM) hybridisation at 30° C. followed by direct addition of streptavidin-gold conjugate (500 pg/μl)

FIG. 16: Assembly of an array which is 2 mm×2 mm in size and contains 10×10=100 probes.

The numbers 1-10 each stand for an oligonucleotide probe which has been applied 8 to 10 times to the array; “M” stands for a mixture of markers, which includes an immobilised biotin-labelled oligonucleotide at a concentration of 10 μM.

Hybridised target 3 was complementary to probes 5, 7 and 9 (each a perfect match) and to the probes 6, 8 and 10 (each a mismatch with one insertion).

FIG. 17: Probe array after hybridisation and conjugation (for array assembly see FIG. 16).

The pictures from left to right were taken 5 min, 10 min and 20 min after the start of the silver enhancement.

upper: target 3 (1 nM) hybridisation

lower: target 3 (100 nM) hybridisation

FIG. 18: Signal intensities after silver enhancement for different times at two different target concentrations (cf. the pictures in FIG. 17)

FIG. 19a (upper) and b (lower): Time course of the silver enhancement, with probes 5 and 6 as examples (cf. FIGS. 16 to 18).

Each point of measurement for the probe is the mean of 7 to 10 repeated spots.

pm: Perfect match probe (probe no. 5)

mm: Mismatch probe (probe no. 6)

a: Target 3 (1 nM): hybridisation at 55° C.; streptavidin-gold conjugate (125 pg/μl)

b: Target 3 (100 nM): hybridisation at 55° C.; streptavidin-gold conjugate (125 pg/μl)

FIG. 20: Calculated linear regression lines after hybridisation with target 3 (100 nM and 1 nM) for two selected probes in the probe array, valid between 4 and 13 min after the start of the silver enhancement.

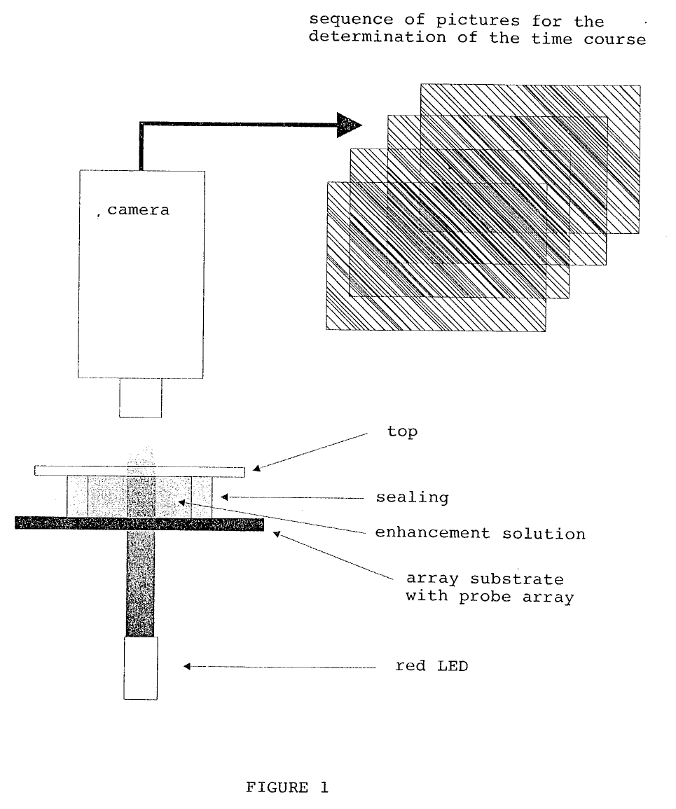

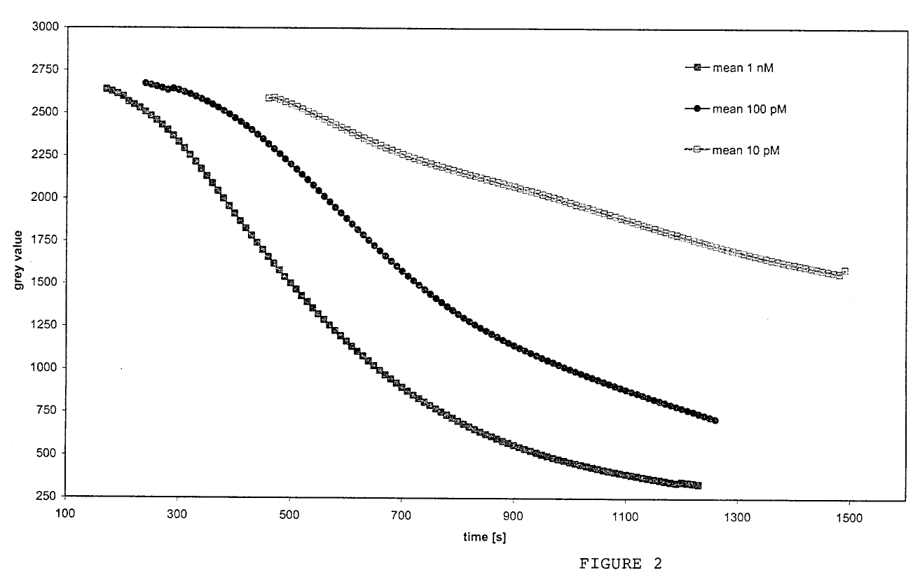

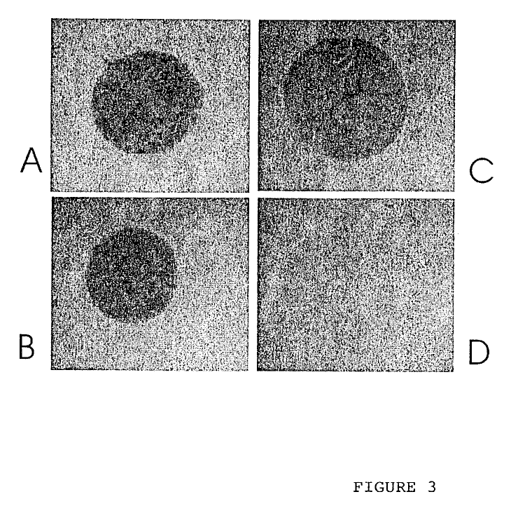

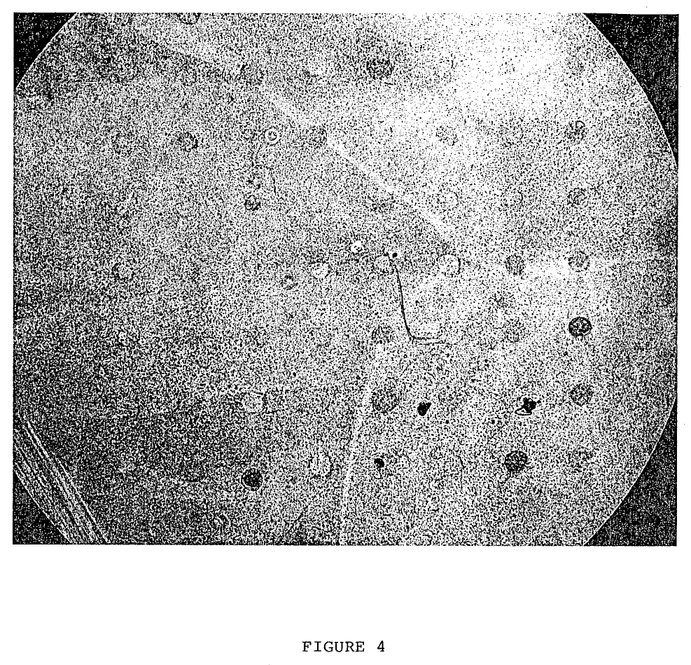

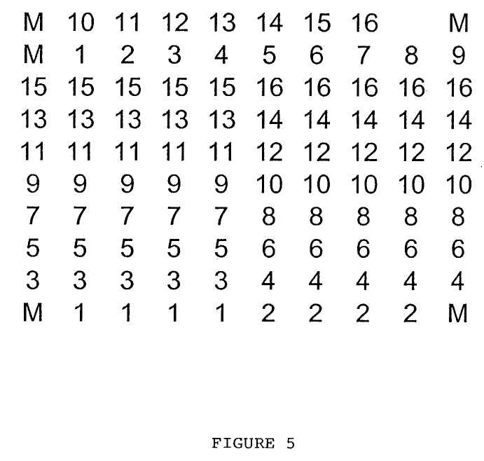

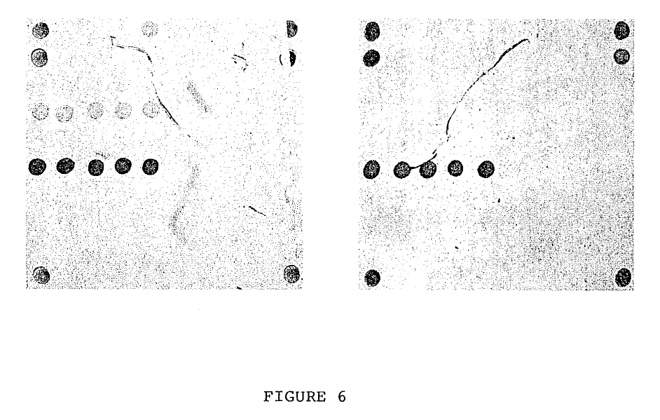

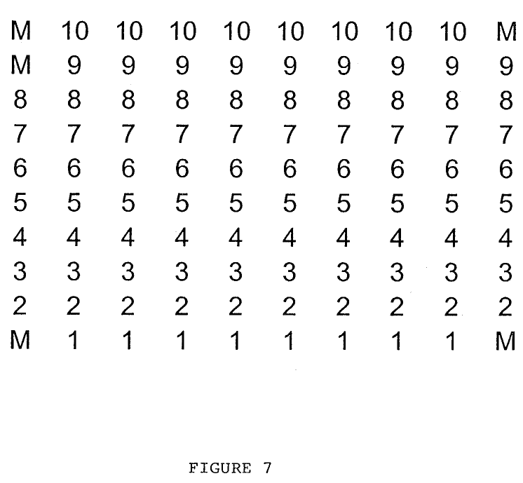

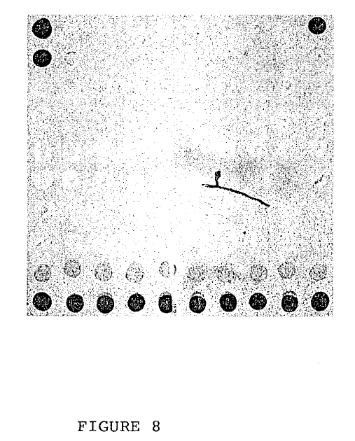

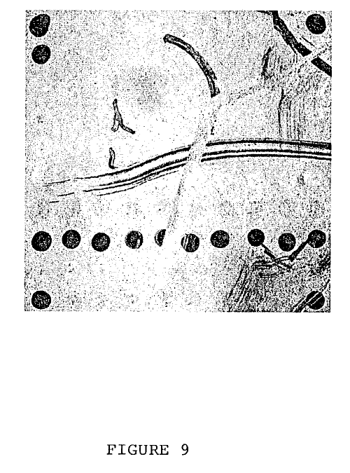

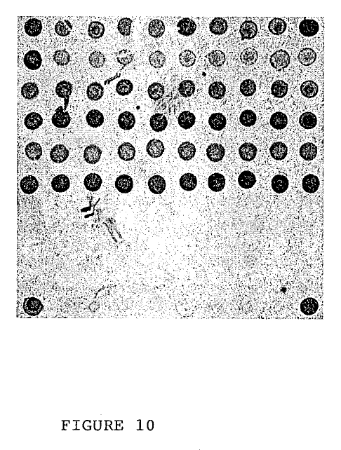

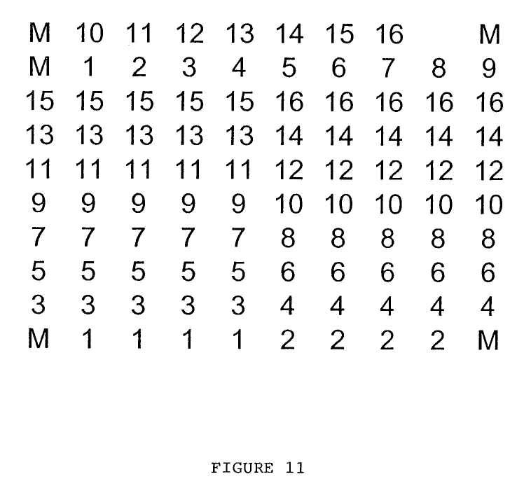

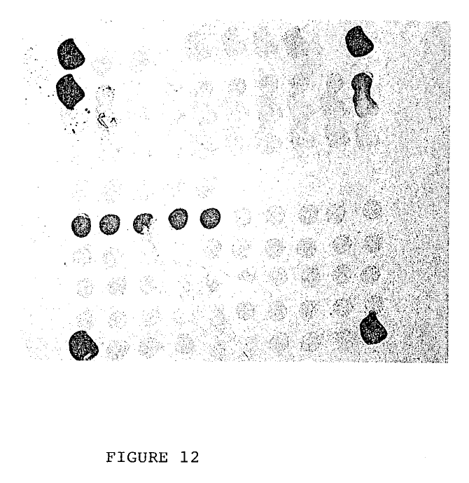

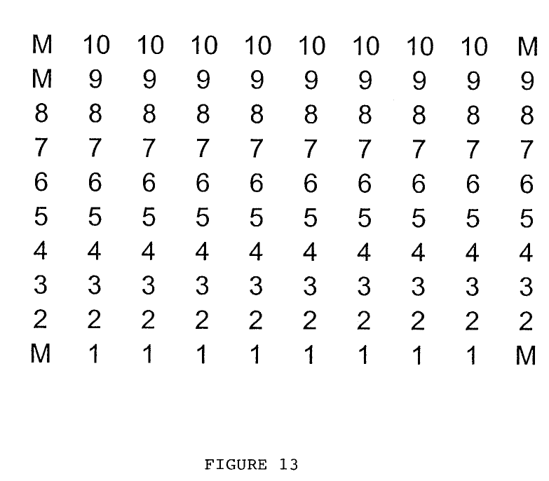

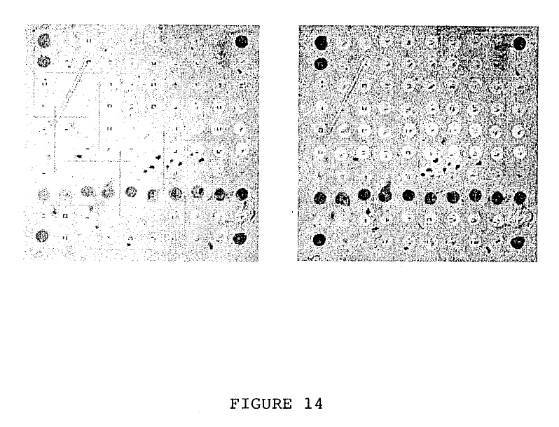

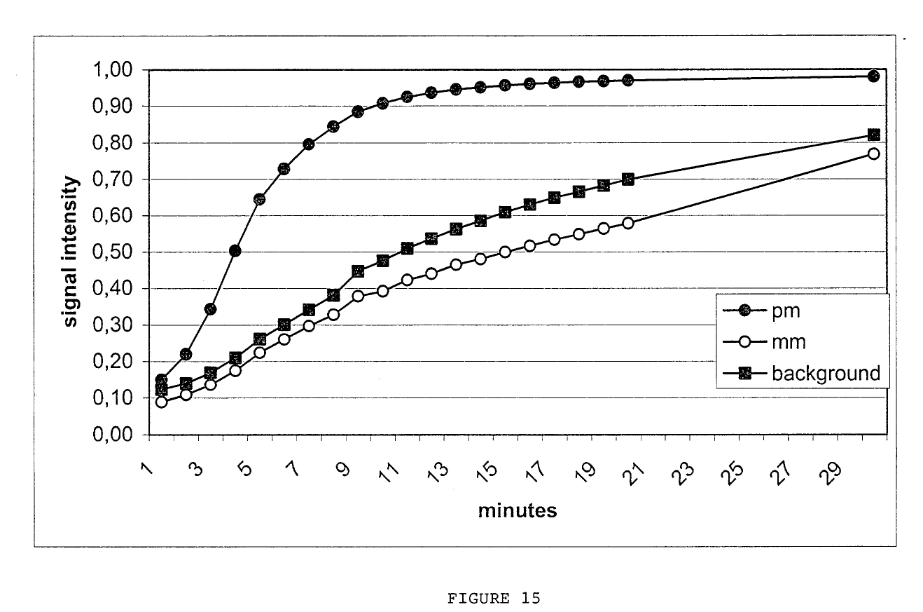

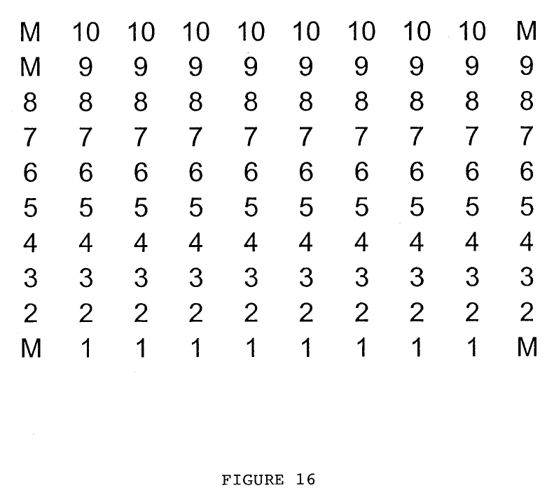

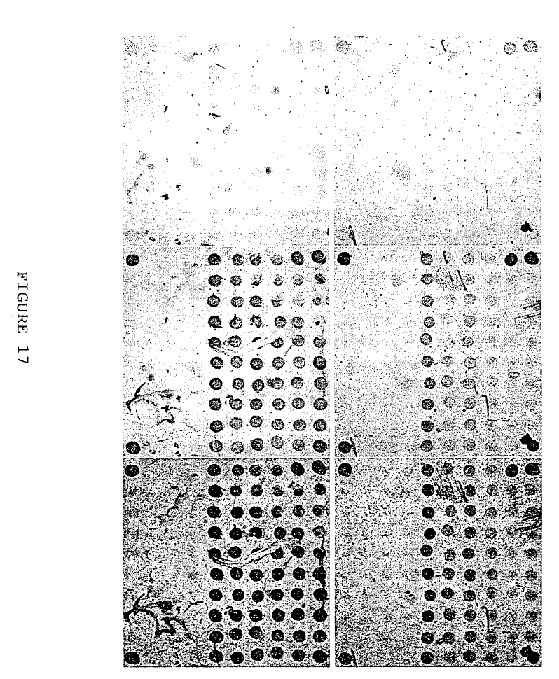

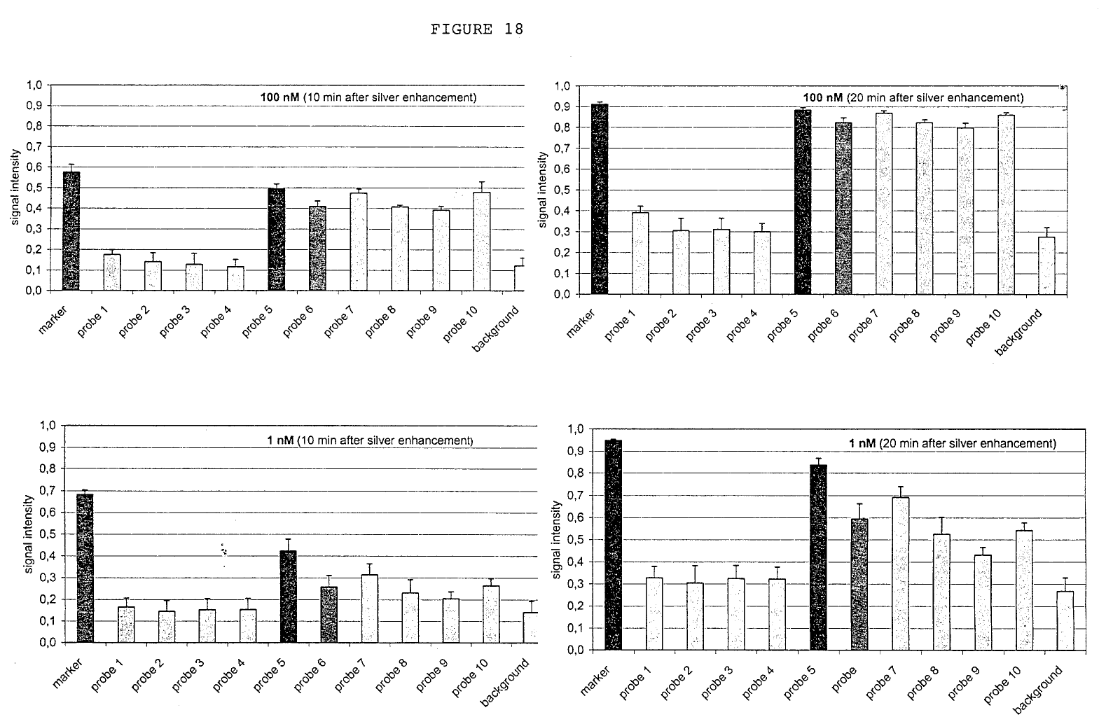

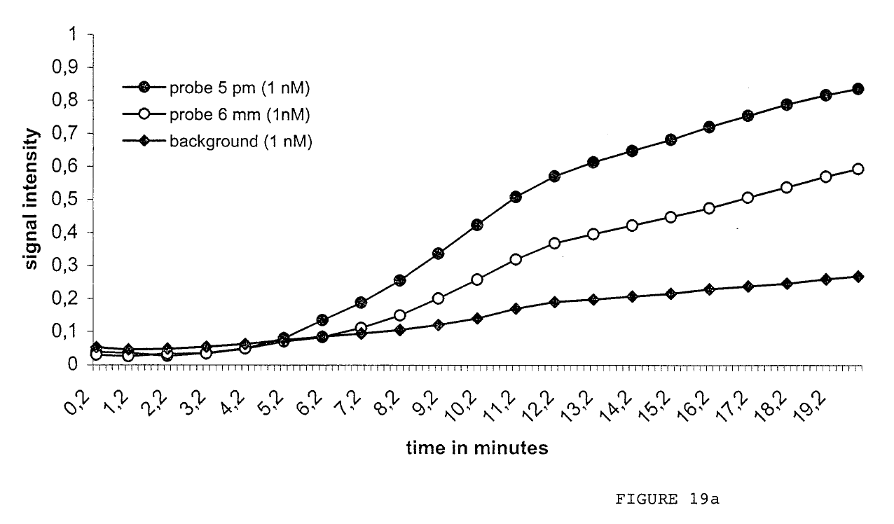

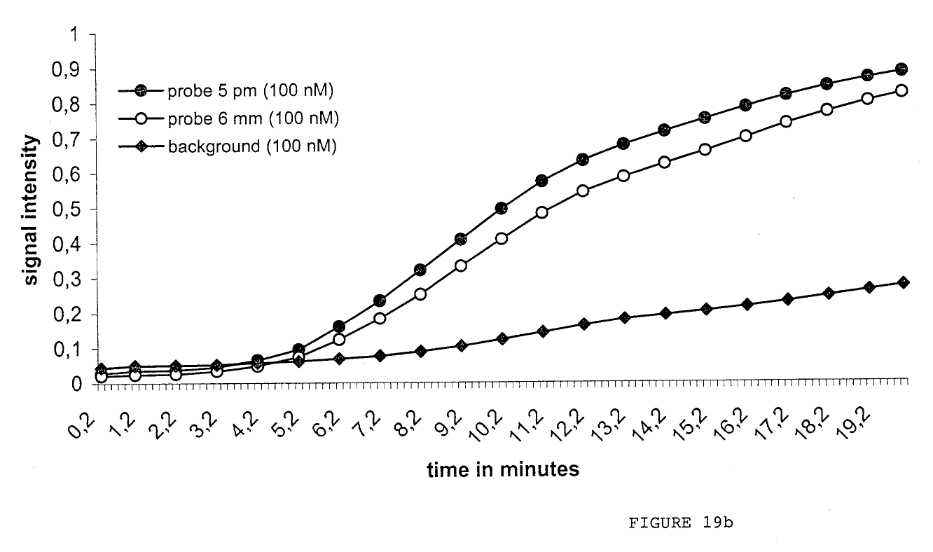

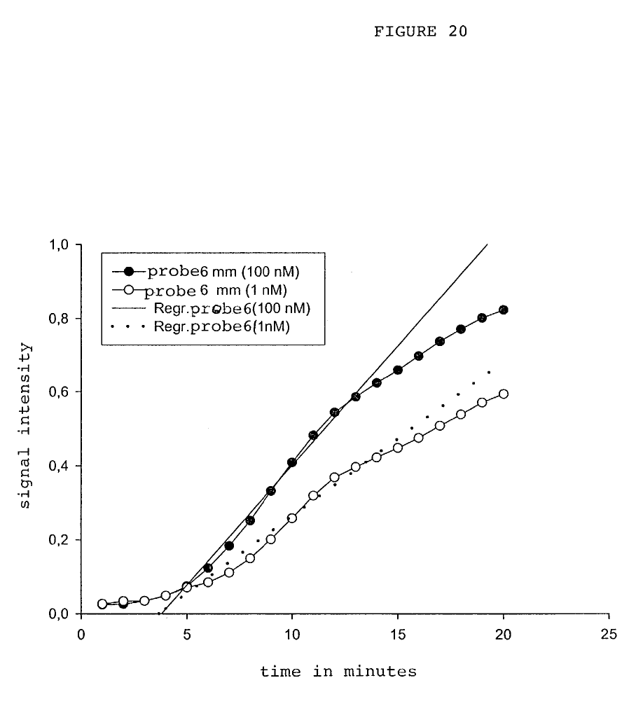

1. A method for the qualitative and/or quantitative detection of targets in a sample by molecular interactions between probes and targets on probe arrays, comprising the following steps:
a) Preparation of a probe array, with probes immobilised at defined sites;
b) Interaction of the target with the probes arranged on the probe array;
c) Performance of a reaction which leads to a precipitate on array elements on which an interaction has occurred;
d) Detection of the time course of the formation of the precipitate on the array elements in the form of signal intensities;
e) Determination of a virtual signal intensity on the basis of a curve function which describes the formation of the precipitate as a function of time.

2. The method according to claim 1,
characterised by the virtual signal intensity for an array element being determined in dependency on the gradient of a regression line which describes the formation of the precipitate as a function of time.

3. The method according to claim 2,
characterised by the regression line being determined in the phase of the exponential increase in formation of the precipitation with time on the array element.

4. The method according to claims 2 or 3,
characterised by the virtual signal intensity for an array element being determined by multiplication of the detected signal intensity at a defined time point, preferably of the signal intensity of the last measurement, with the gradient of the regression line which has been determined for the array element and with the time of measurement up to this defined time point.

5. The method according to any of the preceding claims,
characterised by a reference target being present in the sample at a known concentration which interacts with at least one probe in the probe array.

6. The method according to any of the preceding claims,
characterised by the signal intensities for detection of the formation of precipitate on the array elements being recorded at least each minute, preferably every 30 seconds, more preferably every 10 seconds.

7. The method according to any of the preceding claims,
characterised by the reaction which lead to the formation of a precipitate on the array elements being the conversion of a soluble substrate to an insoluble product in the presence of a catalyst which is coupled to the target.

8. The method according to claim 7,
characterised by the catalyst being an enzyme.

9. The method according to claims 7 or 8,
characterised by the enzyme being selected from the group consisting of horseradish peroxidase, alkaline phosphatase and glucose oxidase.

10. The method according to any of the claims 7 to 9,
characterised by the soluble substrate being selected from the group consisting of 3,3′-diaminobenzidine, 4-chlor-1-naphthol, 3-amino-9-ethylcarbazole, p-phenylendiamine-HCl/pyrocatechol, 3,3′,5,5′-tetramethylbenzidine, naphthol/pyronine, bromchlorindoylphosphate, nitrotetraazolium blue and phenazine methosulphate.

11. The method according to any of the claims 1 to 6,
characterised by the reaction which leads to the formation of a precipitate on the array elements being the conversion of a soluble substrate into a metallic precipitate.

12. The method according to claim 11,
characterised by the reaction which leads to the formation of a precipitate on the array elements being the chemical reduction of a silver compound, preferably silver nitrate, silver lactate, silver acetate or silver tartrate, to elemental silver.

13. The method according to claim 12,
characterised by the reductant being selected from the group consisting of formaldehyde and hydroquinone.

14. The method according to any of the claims 11 to 13,
characterised by the conversion of a soluble substrate into a metallic precipitate taking place in the presence of metal clusters or colloidal metal particles which are coupled to the targets.

15. The method according to claim 14,
characterised by the conversion of a soluble substrate into a metallic precipitate taking place in the presence of gold clusters or colloidal gold particles.

16. The method according to any of the claims 11 to 13,
characterised by the conversion of a soluble substrate into a metallic precipitate taking place in the presence of polyanions coupled to the targets.

17. The method according to any of the claims 7 to 16,
characterised by the catalysts or colloidal metallic particles or polyanions being coupled to the target, before, during or after the interaction with the probes.

18. The method according to any of the claims 7 to 17,
characterised by the coupling of the enzymes or metal clusters or colloidal metal particles or polyanions to the targets being carried out directly or through anchor molecules which are coupled to the targets.

19. The method according to claim 18,
characterised by the anchor molecule being selected from the group consisting of streptavidin or an antibody.

20. The method according to any of the claims 1 to 6,
characterised by the reaction which leads to the formation of a precipitate on the array elements being the binding of a specific binding partner to an anchor molecule which is coupled to the targets.

21. The method according to claim 20,
characterised by the binding partner/anchor molecule pair being selected from the group consisting of biotin/avidin or streptavidin or anti-biotin antibodies, digoxigenin/anti-digoxigenin immunoglobulin, FITC/anti-FITC immunoglobulin and DNP/anti-DNP immunoglobulin.

22. The method according to any of the preceding claims,
characterised by the target being directly supplied with a label.

23. The method according to any of the claims 1 to 21,
characterised by the labelling of the target being carried out with sandwich reactions or with sandwich hybridisation with the probes which interact with the targets and a labelled compound.

24. The method according to any of the claims 1 to 21,
characterised by the labelling of the target being carried out by adding a homopolymeric nucleotide sequence to the target, with formation of a continuous sequence, followed by sandwich hybridisation with a labelled oligonucleotide which is complementary to the homopolymeric nucleotide sequence.

25. The method according to any of the preceding claims,
characterised by the interaction between the target and the probe being a hybridisation between two nucleotide sequences.

26. The method according to any of claims 1 to 24,
characterised by the interaction between target and probe being an interaction between an antigenic structure and the corresponding antibody or a hypervariable region thereof.

27. The method according to any of the preceding claims,
characterised by the interaction between the target and the probe being a reaction between a receptor and the corresponding ligand.

28. The method according to any of the preceding claims,
characterised by the detection of the presence of a precipitate on an array element being carried out by reflection, absorption or diffusion of a light beam, preferably a laser beam or a light-emitting diode.

29. The method according to any of the claims 1 to 27,
characterised by the detection of the presence of a precipitate on an array element being carried out electrically.

30. The method according to claim 29,
characterised by the electrical detection being carried out by measurements of conductivity, capacity or potential.

31. The method according to any of the claims 1 to 27,
characterised by the presence of a precipitate on an array element being detected by autoradiography, fluorography and/or indirect autoradiography.

32. The method according to any of the claims 1 to 27,
characterised by the presence of a precipitate on an array element being detected by scanning electron microscopy, electron probe microanalysis (EPMA), magneto-optic Kerr microscopy, magnetic force microscopy (MFM), atomic force microscopy (AFM), measurement of the mirage effect, scanning tunnelling microscopy (STM), and/or ultrasound reflection tomography.

33. The method according to any of the preceding claims, including the following steps:
Detection of the time course of the formation of the precipitate on the array elements by taking pictures with a camera;
Conversion of the analog information contained in the pictures into the digital form;
Calculation of a virtual signal intensity for each array element on the basis of a curve function which describes the precipitate formation as a function of time;
Conversion of the virtual signal intensities into an artificial image which describes the virtual signal intensities of all array elements.

34. A device to perform the method according to any of the claims 1 to 33, including:
a) an array substrate with probe array,
b) a reaction chamber,
c) a device for the detection of a precipitate on an array element on which an interaction between targets and probes has occurred, and
d) a computer, which is programmed to:
Collect the signal intensities recorded by the detection device;
Guarantee the processing of the successively recorded signal intensities, so that the time course of the formation of the precipitate on an array element is determined and a virtual signal intensity is determined on the basis of a curve function which describes the formation of the precipitate as a function of time;
Guarantee, if required, the conversion of the virtual signal intensities into an analog picture.

35. The device according to claim 34,
characterised by the detection device being a camera.

36. The device according to claim 35,
characterised by the camera being a CCD or a CMOS camera.

37. The device according to any of the claims 34 to 36,
characterised by the device also including a light source.

38. The device according to claim 37,
characterised by the light source being selected from the group consisting of a laser, a light-emitting diode (LED), and a high pressure lamp.

39. The device according to any of the claims 24 to 38,
characterised by the device being present as a highly integrated autonomous unit.
